(19) United States
(12) Patent Application Publication
(10) Pub. No.: US 2024/0366638 A1
(43) Pub. Date: Nov. 7, 2024

(54) PSILOCYBIN ANALOGS FOR TREATING PSYCHOLOGICAL DISORDERS

(71) Applicant: SYNAPTIVE THERAPEUTICS LLC, PEARLAND, TX (US)

(72) Inventors: Matthew Halpert, Pearland, TX (US); John Hartenbach, Pearland, TX (US)

(21) Appl. No.: 18/572,829

(22) PCT Filed: Jun. 27, 2022
(86) PCT No.: PCT/US2022/035181

§ 371 Date: Dec. 21, 2023

Related U.S. Application Data
(60) Provisional application No. 63/215,411, filed on Jun. 25, 2021.

(51) Int. Cl. A61K 31/675; A61K 31/4045; A61P 25/22; A61P 25/24

(52) U.S. Cl. CPC A61K 31/675; A61K 31/4045; A61P 25/22; A61P 25/24

(57) ABSTRACT

Compositions comprising a psilocybin analog and methods of using the compositions for treating a psychological disorder in a subject in need thereof are disclosed. The psilocybin analog can be used at concentrations that provide the therapeutic benefit without the non-therapeutic psychoactive effects.

### CROSS-REFERENCE TO RELATED APPLICATIONS

The present application is a National Stage application of International Application No. PCT/US22/35181, filed Jun. 27, 2022, and published as WO/2022272176 on Dec. 29, 2022, claims priority from Provisional Application No. 63/215,411, filed Jun. 25, 2021, the entire contents of each of which is hereby incorporated by reference.

### FIELD OF THE INVENTION

The present disclosure provides compositions comprising a psilocybin analog and methods of using the compositions for treating a neurological disorder.

### BACKGROUND OF THE INVENTION

It has been reported that about one in five American adults, an alarming 20% of the adult U.S. population, suffers from some form of mental illness or psychological disorder. It has also been reported that nearly half of the U.S. population, i.e., 46%, will have a diagnosable mental health condition at some point in their life, and that nearly half of those persons will develop these conditions by the age of fourteen. Diagnosed mental health conditions include a variety of psychological disorders including anxiety, addiction/substance abuse, bipolar disorder, various forms of depression, post-traumatic stress disorder, more commonly known as PTSD, schizophrenia, and suicidal thoughts among others. In more severe cases, a person may experience combinations of several of these psychological disorders. Treatment of psychological disorders currently consist of medication regimens, therapy sessions, or combinations of the two. Some of the more common types of prescribed medications include, but are not limited to, antianxiety medications; antidepressant medications to improve moods; antipsychotic medications to treat disordered thought patterns and altered perceptions, and/or mood-stabilizing medications. Given the wide range of medications typically prescribed for various psychological disorders, the efficacy of a particular medication for a particular disorder for a particular patient is highly variable. Often, identifying a safe and effective treatment for each patient is a hit or miss strategy depending on a patient's reaction to a particular medication or dosing regimen, which can result in a patient being over, under or improperly treated for potentially extended periods of time causing unnecessary discomfort, and perhaps worse.

In addition, there are a wide range of side effects, from mild to severe, which have been observed in patients taking these various medications. As one example, a common class of antidepressants include selective serotonin reuptake inhibitors, or SSRIs. Numerous negative side effects are associated with SSRIs including, but not limited to: feeling agitated, shaky or anxious; feelings of being sick; indigestion; diarrhea or constipation; loss of appetite and weight loss; dizziness; blurred vision; dry mouth; excessive sweating; sleeping problems, including insomnia or drowsiness; headaches; reduced sex drive; difficulty achieving orgasm during sex or masturbation; and in men, difficulty obtaining or maintaining an erection; i.e., erectile dysfunction. Serotonin-norepinephrine reuptake inhibitors, or SNRIs, are another class of antidepressants that are often prescribed to treat depression as well as anxiety. Common side effects associated with SNRIs essentially parallels the side effects noted above for SSRIs. Other classes of medications often prescribed to treat persons with psychological disorders include tricyclic antidepressants and benzodiazepines, each coming with their own set of similar negative side effects, with the addition of potential addiction and/or overdose in the case of benzodiazepines.

Accordingly, there is an established need for a solution to the foregoing problems and shortcomings in the present state of the art with regard to methods of treatment for psychological disorders with medication.

### SUMMARY OF THE INVENTION

One aspect of the instant disclosure encompasses a method of treating a psychological disorder in a subject in need thereof. The method comprises administering to the subject a therapeutically effective amount of a composition comprising a psilocybin analog. Administering the composition can comprise administering a unit dose of the composition to the subject, wherein the unit dose comprises a therapeutically effective amount of the psilocybin analog. The psilocybin analog can be administered at a sub-hallucinogenic concentration.

The psilocybin analog can be a psilocybin analog of Table 1. In some aspects, the psilocybin analog is a psilocybin analog of Table 2. In some aspects, the psilocybin analog is baeocystin. In some aspects, the psilocybin analog is baeocystin and the and the therapeutically effective amount of baeocystin ranges from about from about 9.5 mg/kg to about 10.5 mg/kg, from about 25 mg/kg to about 35 mg/kg, from about 45 mg/kg to about 55 mg/kg, or from about 165 mg/kg to about 175 mg/kg body weight of the subject. In other aspects, the psilocybin analog is norpsilocin. In other aspects, the psilocybin analog is norpsilocin and the therapeutically effective amount of baeocystin ranges from about 0.25 mg/kg to about 0.35 mg/kg or from about 2.5 mg/kg to about 3.5 mg/kg body weight of the subject. In yet other aspects, the psilocybin analog is baeocystin, norpsilocin, or a combination of baeocystin and norpsilocin. In some aspects, the psilocybin analog is baeocystin, norpsilocin, or a combination of baeocystin and norpsilocin and the therapeutically effective amount of baeocystin, norpsilocin, or a combination of baeocystin and norpsilocin ranges from about 0.3 mg/kg to about 3.0 mg/kg body weight of the subject.

Another aspect of the instant disclosure encompasses a pharmaceutical composition for treating inflammation or a psychological disorder. The composition comprises a psilocybin analog. The psilocybin analog can be a psilocybin analog of Table 1. In some aspects, the psilocybin analog is a psilocybin analog of Table 2. In some aspects, the psilocybin analog is baeocystin. In other aspects, the psilocybin analog is norpsilocin. In yet other aspects, the psilocybin analog is baeocystin, norpsilocin, or a combination of baeocystin and norpsilocin.

Yet another aspect of the instant disclosure encompasses a kit for treating a psychological disorder comprising one or more pharmaceutical compositions comprising a psilocybin analog. The composition can be as described herein above.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 are chemical structures of serotonin and serotonin-like psilocin and N,N-Dimethyltryptamine (DMT).

FIG. 2 is a diagram depicting metabolism of psilocybin.

FIG. 3 is a plot showing the effects of baeocystin on center time in the open field test. Mice were administered various doses of baeocystin and locomotor activity collected over a 30 min period. Time in the center zone of the open field chamber are presented. There were no significant difference between groups (Main Effect, F(6, 62)=1.096, P=0.374).

FIG. 4 is a plot showing the effect of baeocystin on locomotor activity. Mice were administered various doses of baeocystin, and locomotor activity collected over a 30 min period. Statistical analysis showed significant differences between the highest dose of baeocystin and all other groups. (Main Effect: F(6, 62)=7.369, P<0.0001; *P<0.05, **P<0.01, ***P<0.001, ****P<0.0001).

FIG. 5 is a plot showing the effects of baeocystin in the elevated plus maze assay. Mice were administered various doses of baeocycstin then placed on the EPM apparatus for 5 minutes and time spent on the open arms obtained. Statistical analysis showed the highest dose of baeocystine increased time spent on the open arm compared to saline. (Main Effect: F(6, 62)=7.183, P<0.0001, ****P<0.0001).

FIG. 6 is a plot showing the effects of baeocystin in the tail suspension test. Mice were administered various doses of baeocystin then suspended by their tails from a metal shelf for 6 minutes and their activity recorded. Statistical analysis showed significant differences between the two highest doses of baeocystin and other groups. (Main Effect: F(6, 63)=5.427, P=0.0001; *P<0.05, ***P<0.001).

FIG. 7 is a plot showing the effects of baeocystin in the forced swim test. Mice were administered various doses of baeocystin and placed in the forced swim cylinder full of water for 6 minutes and activity recorded. Statistical analysis revealed no significant differences between groups. (Main Effect: F(6, 63)=1.727, P=0.1293).

FIG. 8 is a plot showing the effects of norbaeocystin on center time in the open field test. Mice were administered various doses of norbaeocystin and locomotor activity collected over a 30 min period. Time in the center region of the open field chamber are presented. There was a significant difference between diazepam and the highest dose of norbeaocystin (Main Effect: F(4, 44)=3.399, P=0.0166, *P<0.05).

FIG. 9 is a plot showing the effect of norbaeocystin on locomotor activity. Mice were administered various doses of norbaeocystin and locomotor activity collected over a 30 min period. Statistical analysis showed a trend towards a difference between norbaeocystin 1.0 vs. 3.0 mg/kg (Main Effect: F(4, 44)=2.885, P=0.0332, P=0.060).

FIG. 10 is a plot showing the effects of norbaeocystin in the elevated plus maze assay. Mice were administered various doses of norbaeocycstin then placed on the EPM apparatus for 5 minutes and time spent on the open arms obtained. Statistical analysis showed no differences between groups (Main Effect: F (4, 44)=0.7858, P=0.5406).

FIG. 11 is a plot showing the effects of norbaeocystin on the tail suspension test. Mice were administered various doses of norbaeocystin then suspended by their tails from a metal shelf for 6 minutes and their activity recorded. Statistical analysis showed significant differences between saline and ketamine and all doses of norbaeocystin (Main Effect: F(4, 45)=9.966, P<0.0001; *P<0.05, ***P<0.001).

FIG. 12 is a plot showing the effects of norbaeocystin on the forced swim test. Mice were administered various doses of norbaeocystin and placed in the forced swim cylinder full of water for 6 minutes and activity recorded. Statistical analysis revealed no significant differences between groups (Main Effect: F(4, 45)=1.777, P=0.1501).

FIG. 13 is a plot showing the effects of aeruginascin on center time in the open field test. Mice were administered various doses of aeruginascin and locomotor activity collected over a 30 min period. Time in the center region of the open field chamber are presented. There were no significant differences between groups (F(4, 44)=1.974, P=0.1152).

FIG. 14 is a plot showing the effects of aeruginascin on locomotor activity. Mice were administered various doses of aeruginascin and locomotor activity collected over a 30 min period. Statistical analysis showed a significant difference between aeruginascin 0.3 and 1.0 mg/kg (Main Effect: F(4, 44)=2.888, P=0.0330, *P<0.05).

FIG. 15 is a plot showing the effects of aeruginascin on the elevated plus maze assay. Mice were administered various doses of aeruginascin then placed on the EPM apparatus for 5 minutes and time spent on the open arms obtained. Statistical analysis showed no differences between groups F(4, 44)=2.402, P=0.0641).

FIG. 16 is a plot showing the effects of aeruginascin on the tail suspension test. Mice were administered various doses of aeruginascin then suspended by their tails from a metal shelf for 6 minutes and their activity recorded. Statistical analysis showed significant differences between saline and ketamine and all doses of aeruginascin (Main Effect: F(4, 45)=14.95, P<0.0001; **P<0.001, ****P<0.0001).

FIG. 17 is a plot showing the effects of aeruginascin on the forced swim test. Mice were administered various doses of aeruginascin and placed in the forced swim cylinder full of water for 6 minutes and activity recorded. Statistical analysis revealed significant differences between ketamine and aeruginascin 0.3 and 3.0 mg/kg doses (F(4, 44)=3.637, P=0.0121, *P<0.05).

FIG. 18 is a plot showing the effects of norpsilocin on center time in the open field test. Mice were administered various doses of norpsilocin and locomotor activity collected over a 30 min period. Time in the center region of the open field chamber are presented. There were highly significant differences between the highest dose of norpsilocin and all other groups (Main Effect: F(4, 44)=13.95, P<0.0001; *P<0.05, **P<0.01, ***P<0.001, ****P<0.0001).

FIG. 19 is a plot showing the effects of norpsilocin on locomotor activity. Mice were administered various doses of norpsilocin and locomotor activity collected over a 30 min period. Statistical analysis revealed significant differences between diazepam and norpsilocin 1.0 and 3.0 mg/kg (F(4, 44)=4.786, P=0.0027, *P<0.05, **P<0.01).

FIG. 20 is a plot showing the effects of norpsilocin on the elevated plus maze assay. Mice were administered various doses of norpsilocin then placed on the EPM apparatus for 5 minutes and time spent on the open arms obtained. Statistical analysis showed no differences between groups (Main Effect: F(4, 44)=2.188, P=0.859).

FIG. 21 is a plot showing the effects of norpsilocin on the tail suspension test. Mice were administered various doses of norpsilocin then suspended by their tails from a metal shelf for 6 minutes and their activity recorded. Statistical analysis showed significant differences between ketamine and all doses of norpsilocin (Main Effect: F(4, 45)=5.786, P=0.0008, **P<0.001).

FIG. 22 is a plot showing the effects of norpsilocin on the forced swim test. Mice were administered various doses of norpsilocin and placed in the forced swim cylinder full of water for 6 minutes and activity recorded. Statistical analysis revealed no differences between any groups (Main Effect: F(4, 45)=1.643, P=0.1800)

### DETAILED DESCRIPTION

The present disclosure is based in part on the discovery that psilocybin analogs can have different psychoactive properties. As used herein, the term “psychoactive properties or effects” refers to any change in the function of the nervous system resulting in alterations in perception, mood, consciousness, cognition, or behavior. Compounds comprising psychoactive properties can be used medically to provide a therapeutic benefit, recreationally, or spiritually; to purposefully improve performance or alter one's consciousness; as entheogens for ritual, spiritual, or shamanic purposes, or for research.

The inventors surprisingly discovered that individual psilocybin analogs can have a different psychoactive effect when administered to a subject in pure form in isolation from other psylocibin analogs that are normally present in magic mushrooms or from metabolites of the same compound. Importantly and surprisingly, the inventors also discovered that some of the psylocibin analogs in fact can worsen a psychological disorder when administered to a subject. Additionally, when a psilocybin analog of the instant disclosure exhibits psychoactive activity in addition to a beneficial therapeutic activity, the psilocybin analog can be administered at concentrations sufficiently low to provide a therapeutic effect without the non-therapeutic psychoactive effects.

### I. Composition

One aspect of the present disclosure encompasses pharmaceutical compositions comprising a psilocybin analog for treating psychological disorders.

### (a) Psilocybin Analogs

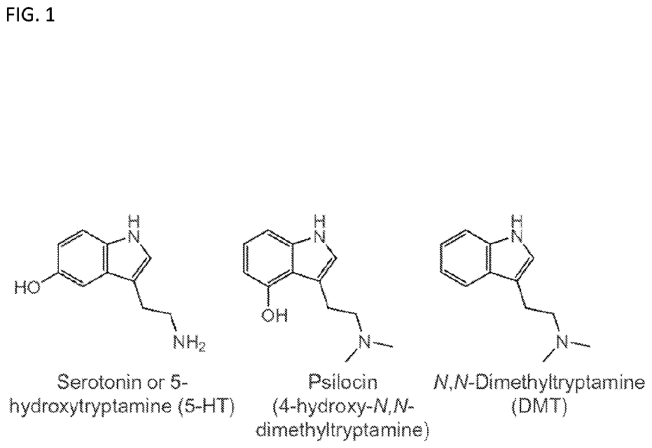

A composition of the instant disclosure comprises a psilocybin analog. As used herein, the term “psilocybin analog” refers to psilocybin, a metabolite thereof, an analog thereof, a derivative thereof, or a psilocybin-like molecule. Psilocybin analogs are tryptamine compounds comprising an indole ring linked to an ethylamine substituent. Psilocybin is chemically related to the amino acid tryptophan and is structurally similar to the neurotransmitter serotonin (FIG. 1).

In some aspects, a psilocybin analog of the instant disclosure is a tryptamine compound of Formula I. The groups designated with an R can be varied as needed to define specific molecules in this chemical family.

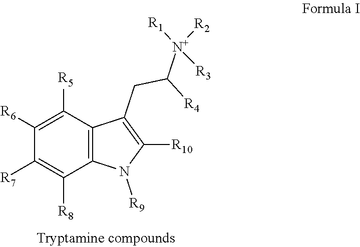

In some aspects, a psilocybin analog of the instant disclosure is a tryptamine compound as set forth in Table 1, or any combination thereof.

TABLE 1
Psilocybin
Psilocin
4-Hydroxytryptamine
O-Methylpsilocin
4-Hydroxy-N,N diethyltryptamine
3-(2-Pyrrolidin-1-yl-ethyl)-1H-indol-4-ol
Indol-6-OL, 3-(2-(dimethylamino)ethyl)-
4-Hydroxy-N-methyltryptamine 4-phosphate
4-Hydoxytryptamine 4-phosphate
3-(2-Dimethylamino-propyl)-1H-indol-4-ol
4-Hydroxy-N,N-diisopropyltryptamine
4-Hydroxy-N-methyl-N-ethyltryptamine
4-Hydroxy-N-methyl-N-isopropyltryptamine
[3[2-(Dimethylamino)ethyl]-1-methylindol-4-yl] dihydrogen phosphate
Ethocybin
3-(3-Dimethylamino-propyl)-1H-indol-4-ol
3-Dimethylaminomethyl-1H-indol-4-ol
[1-Butyl-3-[2-(dimethylamino)ethyl]indol-4-yl] dihydrogen phosphate
2-(4-Hydroxy-1H-indol-3-yl)ethanaminium
342-(Dimethylazaniumyl)ethyl]-1H-indol-4-yl phosphate
342-(4-Methyl-piperazin-1-yl)-ethyl]-1H-indol-4-ol
Meprocin
3-[2-(Methylamino)ethyl]-1 H-indol-4-ol
3-(2-Dimethylamino-ethyl)-6-fluoro-1H-indol-4-ol
3-(1-Methyl-pyrrolidin-3-yl)-1H-indol-4-ol
Norbaeocystin
Baeocystin
[3-[2-(Trimethylazaniumyl)ethyl]-1 H-indol-4-yl] phosphate
N,N-Diallyl-4-hydroxytryptamine
Aeruginascin
n,n-Diisopropyl-4-hydroxytryptamine hydrochloride
Dipropyl-4-hydroxytryptamine
3-[(2R)-2-Aminopropyl]-1H-indol-4-ol
3-[(2S)-2-Aminopropyl]-1H-indol-4-ol
(R)-3-((1-Methylpyrrolidin-2-YL)methyl)-1H-indol-4-OL
3-Methyl-1H-indol-4-ol
O-Benzyl Psilocybin
3-(2-Methylbut-3-en-2-yl)-1 H-indol-4-ol
1 H-Indol-4-olate
2-(4-Dibenzylphosphoryloxy-1H-indol-3-yl)-N,N-dimethylethanamine
4-Dibenzylphosphoryloxy-3-(2-piperidin-1-ylethyl)-1H-indole
1H-Indol-4-yl phosphate
Tris[[342-(dimethylamino)ethyl]-1H-indol-4-yl]oxy]-hydroxyphosphanium
3-(2-Azidoethyl)-1H-indol-4-ol
3-(4-Azidooxy-1H-indol-3-yl)-N,N-dimethylpropan-1-amine
4-[Azido(methyl)phosphoryl]oxy-1H-indole
[342-(Dimethylamino)ethyl]-1H-indol-4-yl] phosphate
[3[2-[Benzyl(dimethyl)azaniumyl]ethyl]-1H-indol-4-yl] phosphate
4-Azidooxy-3-methyl-1H-indole
1H-Indol-4-yl sulfate
2-[1H-Indol-3-yloxy(methoxy)phosphoryl]oxyethyl-trimethylazanium
[3-[2-(Dimethylamino)ethyl]-1 H-indol-4-ylloxy-methylphosphinic acid
2-(4-Dihydroxyphosphanyloxy-1H-indol-3-yl)ethyl-trimethylazanium
[3-(3-Methylbutyl)-1H-indol-4-yl] phosphono hydrogen phosphate
2-(4-Dimethylphosphoryloxy-1H-indol-3-yl)-N,N-dimethylethanamine
244-[Hydroxy(phenylmethoxy)phosphoryl]oxy-1H-indol-3-yl]ethyl-
dimethylazanium
342-[Amino(methyl)amino]ethyl]-1H-indol-4-ol
Benzyl-[2-[4-[hydroxy(methyl)phosphoryl]oxy-1H-indol-3-yllethyll-
dimethylazanium
3-[2-[Iodo(methyl)amino]ethyl]-1 H-indol-4-ol
Potassium; 3-(2-aminoethyl)-1H-indol-4-olate
N-(1H-Indol-4-yloxy)-2-methylpropan-1-amine; methanol
O-[3-(2-Aminoethyl)-1 H-indol-4-yl]hydroxylamine; fluoromethane
N,N-Dimethyl-2-(4-sulfanyloxy-1H-indol-3-yl)ethanamine; ethane
Ethane; methane; 04342-(methylamino)ethyl]-1H-indol-4-yl]hydroxylamine
3-Ethyl-4-sulfanyloxy-1H-indole; methane
(3-Heptan-3-yl-1H-indol-4-yl) hypofluorite; methane
Ethane,N-methyl-2-(4-sulfanyloxy-1H-indol-3-ypethanamine
Fluoromethane; (3-heptan-3-yl-1H-indol-4-yl) hypofluorite
Fluoromethane; O-[3-[2-(methylamino)ethyl]-1 H-indol-4-yl]hydroxylamine
Methane; (3-pentan-3-yl-1H-indol-4-yl) hypofluorite; propane
Fluoromethane; octane; O-(3-propyl-1H-indol-4-yl)hydroxylamine
[3-(3-Ethylheptyl)-1H-indol-4-yl] hypofluorite; methane
3-(2-Aminoethyl)-1H-indol-4-ol; ethane
3-[2-(Dimethylamino)ethyl]-1 H-indol-4-ol; ethane
1H-Indol-4-yloxyboronic acid
4-Methylsulfanyloxy-1H-indole
2-(5-Phosphorosooxy-1H-indol-3-yl)ethanamine
[3-[2-(Dimethylamino)ethyl]-1 H-indol-4-yl] phosphono hydrogen phosphate
Benzyl-dimethyl-[2-(4-phosphonooxy-1H-indol-3-yl)ethyl]azanium
[342-(Dimethylazaniumypethyl]-1H-indol-4-yl] hydrogen phosphate
[3-[2-(Dimethylamino)ethyl]-1 H-indol-4-yl] dihydrogen phosphate; hydrate
Dibenzyl [342-(dimethylamino)ethyl]-1H-indol-4-yl] phosphate
3-[2-[Ethyl(propyl)amino]ethyl]-1 H-indol-4-ol
[3-(1-Aminoethyl)-1H-indol-4-yl] dihydrogen phosphate
[3-[2-(Dimethylamino)ethyl]-1 H-indol-4-yl] hydrogen phosphate
[1-(Aminomethyl)-3-[2-(methylamino)ethyl]indol-4-yl] hypoiodite
244-(Methoxymethylphosphanyloxy)-1H-indol-3-yl]-N,N-dimethylethanamine
(2S)-2-[[[342-(Dimethylamino)ethyl]-1H-indol-4-yl]oxy-
(methoxymethyl)phosphoryllamino]-2,3-dimethylbutanoic acid
Dibromo 1H-indol-4-yl phosphate
3-[2-(Diethylamino)ethyl]-1 H-indol-6-ol
3-[2-(Ethylamino)ethyl]-1 H-indol-4-ol
1H-Indol-5-yl dihydrogen phosphate
3-[2-(Methylamino)ethyl]-1-propan-2-ylindol-4-ol
1H-Indol-4-yl hypofluorite
[4-(1-Ethyl-3,6-dihydro-2H-pyridin-5-yl)-1H-indol-3-yl] dihydrogen phosphate
3-[2-(Dimethylamino)ethyl]-5-fluoro-1 H-indol-4-ol
3-[2-(Dimethylamino)ethyl]-1 H-indol-4-ol; [3-[2-(dimethylamino)ethyl]-1 H-
indol-4-yl] dihydrogen phosphate
4-Phosphorosooxy-1H-indole
1H-Indol-4-yl dihydrogen phosphate
04342-(Methylamino)ethyl]-1H-indol-6-yl]hydroxylamine
3-(2-Methyl-1,2,3,6-tetrahydropyridin-4-yl)-1H-indol-4-ol
CID 67465946
1H-Indol-4-yloxy(oxo)borane
[3-[2-(Trimethylazaniumyl)ethyl]-1 H-indol-4-yl] hydrogen phosphate
[3[2-(Dimethylamino)ethyl]-7-fluoro-1-methylindol-4-yl] dihydrogen phosphate
[3[2-(Dimethylamino)ethyl]-7-fluoro-1-propylindol-4-yl] dihydrogen phosphate
1-Ethyl-3-[2-(methylamino)ethyl]indol-4-ol; tungsten
CID 58110672
2-(4-Phosphorosooxy-1H-indol-3-yl)ethanamine
N,N-Dimethyl-2-(4-phosphorosooxy-1H-indol-3-yl)ethanamine
4-Hydroperoxy-1 H-indole
1H-Indol-4-yl hypochlorite
3-Butyl-1H-indol-4-ol
1H-Indol-4-yl nitrate
O-(1H-Indol-4-yl)hydroxylamine
1H-Indol-4-yl hypobromite
1H-Indol-4-yl hypoiodite
3-[(1-Methylpyrrolidin-2-yl)methyl]-1H-indol-4-ol
4-Methylperoxy-1 H-indole
N,N-Diethyl-2-(4-methoxy-1H-indol-3-yl)ethanamine
34[2-(Dimethylamino)phenyl]methyl]-1H-indol-4-ol
3-(2-Piperidinoethyl)-1H-indole-4-ol
342-(Dibutylamino)ethyl]-1H-indol-4-ol
4-HO-Dsbt
3-(Aminomethyl)-1H-indol-4-ol
1H-Indol-4-ol, 3-ethyl-
4-Hydroxy-3-(1-methyl-1,2,3,6-tetrahydropyridin-4-yl)-1H-indole
4-Hydroxy-3-(1-benzyl-1,2,3,6-tetrahydropyridin-4-yl)-1H-indole
4-Hydroxy-3-(1,2,3,6-tetrahydropyridin-4-yl)-1H-indole
1-(4-Hydroxy-1H-indol-3-yl)ethyl-trimethylazanium
2-(4-Hydroxy-1H-indol-3-yl)ethyl-trimethylazanium
2-(4-Hydroxy-1H-indol-3-yl)ethyl-trimethylazanium; methyl sulfate
(4-Hydroxy-1H-indol-3-yl)methyl-trimethylazanium
(4-Hydroxy-1H-indol-3-yl)methyl-trimethylazanium; methyl sulfate
Benzyl [3-[2-[benzyl(dimethyl)azaniumyl]ethyl]-1H-indol-4-yl] phosphate
2-[Hydroxy(1H-indol-3-yloxy)phosphoryl]oxyethyl-trimethylazanium
1H-Indol-3-yl (2-(trimethylammonio)ethyl) phosphate
3[2-[Methyl((111C)methyl)amino]ethyl]-1H-indol-4-ol
3-[(2S)-1-Methyl-2-pyrrolidinyl]nethyl]-1H-indole-4-ol
Benzyl-[244-[hydroxy(phenylmethoxy)phosphoryl]oxy-1H-indol-3-yl]ethyl]-
dimethylazanium
1H-Indol-4-ol, 3-(2-aminopropyl)-
342-[Bis(2-methylpropyl)amino]ethyl]-1H-indol-4-ol
3-[2-[Butyl(methyl)amino]ethyl]-1 H-indol-4-ol
3-[2-[Methyl(2-methylpropyl)amino]ethyl]-1H-indol-4-ol
342-[Butan-2-yl(methyl)amino]ethyl]-1H-indol-4-ol
3-[2-[Tert-butyl(methyl)amino]ethyl]-1 H-indol-4-ol
3-[2-[Cyclopentyl(methyl)amino]ethyl]-1H-indol-4-ol
3-[2-(2,6-Dimethylpiperidin-1-yl)ethyl]-1 H-indol-4-ol
3[2-(Dimethylamino)ethyl]-1H-indol-4-ol; phosphoric acid
2-(4-Hydroxy-1H-indol-3-yl)ethyl-dimethylazanium
4-Hydroxy-N-isopropyl-N-methyltryptamine-d4
1-Ethyl-3-[2-(methylamino)ethyl]indol-4-ol
3-[2-(N,2-Dimethylanilino)ethyl]-1H-indol-4-ol
Benzyl [341,1,2,2-tetradeuterio-2-(dimethylamino)ethyl]-1H-indol-4-yl]
hydrogen phosphate
N,N-Dimethyl-2-(4-trimethylsilyloxy-1 H-indol-3-yl)ethanamine
341,1,2,2-Tetradeuterio-2-[ethyl(methyl)amino]ethyl]-1H-indol-4-ol
341,1,2,2-Tetradeuterio-2-(diethylamino)ethyl]-1H-indol-4-ol
341,1,2,2-Tetradeuterio-2-(dimethylamino)ethyl]-1H-indol-4-ol
[341,1,2,2-Tetradeuterio-2-(dimethylamino)ethyl]-1H-indol-4-yl] dihydrogen
phosphate
1H-Indol-4-ol, 3-(3-aminopropyl)-
342-(Methylamino)propan-2-yl]-1H-indol-4-ol
3-Methyl-5-[2-(methylamino)ethyl]-1 H-indol-4-ol
5-(3-Aminopropyl)-3-methyl-1H-indol-4-ol
3-(2-Aminoethyl)-1-methylindol-4-ol
1-Methyl-3-[2-(methylamino)ethyl]indol-4-ol
3-(3-Aminopropyl)-1-methylindol-4-ol
3-(1-Aminopropan-2-yl)-1-methylindol-4-ol
3-(2-Aminopropyl)-1-methylindol-4-ol
3-(Methylaminomethyl)-1H-indol-4-ol
3-[2-(Methylamino)propyl]-1 H-indol-4-ol
3-(2-Aminoethyl)-1-propan-2-ylindol-4-ol
1-Methyl-3-[2-(methylamino)propyl]indol-4-ol
3-(3-Aminopropyl)-1-propan-2-ylindol-4-ol
3-(2-Aminopropyl)-1-propan-2-ylindol-4-ol
3-(4-Piperidinyl)-1H-Indol-4-ol
1-Methyl-3-(1,2,3,6-tetrahydropyridin-4-yl)indol-4-ol
1-(3-Aminopropyl)-3-methylindol-4-ol
3-(2-Aminoethyl)-5-ethyl-1H-indol-4-ol
3-(Fluoromethyl)-1H-indol-4-ol
3-(1-Fluoroethyl)-1H-indol-4-ol
3-(2-Fluoropropan-2-yl)-1H-indol-4-ol
3-(Pyrrolidin-1-ylmethyl)-1H-indol-4-ol
3-(Piperidin-1-ylmethyl)-1H-indol-4-ol
3-(2-Aminoethyl)-1-ethylindol-4-ol
3-(2-Aminoethyl)-1-propylindol-4-ol
3-(2-Aminoethyl)-1-prop-2-enylindol-4-ol
[3[2-(Dimethylamino)ethyl]-1-trimethylsilylindol-4-yl] bis(trimethylsilyl)
phosphate
3-[2-[Hydroxy(methyl)amino]propan-2-yl]-1 H-indol-4-ol
3-[[(3R)-1-Methyl-3-pyrrolidinyl]methyl]-1H-indole-4-ol
3-[[(3S)-1-Methyl-3-pyrrolidinyl]methyl]-1H-indole-4-ol
3-Propan-2-yl-1H-indol-4-ol
342-[Bis(trideuteriomethypamino]-1,1,2,2-tetradeuterioethyl]-1H-indol-4-ol
3-(1-Aminopropan-2-yl)-1-ethylindol-4-ol
3-(2-Aminopropyl)-1-ethylindol-4-ol
3-(3-Aminopropyl)-1-ethylindol-4-ol
3-[(Cyclobutylamino)methyl]-1 H-indol-4-ol
3-[(Cyclopentylamino)methyl]-1 H-indol-4-ol
3-(Azepan-1-ylmethyl)-1H-indol-4-ol
3-[(4-Methylpiperazin-1-yl)methyl]-1H-indol-4-ol
3-(Ethylaminomethyl)-1H-indol-4-ol
3-[(Propan-2-ylamino)methyl]-1 H-indol-4-ol
3-(Ethylaminomethyl)-1-methylindol-4-ol
1-Methyl-3-[(propan-2-ylamino)methyl]indol-4-ol
1-Ethyl-3-(methylaminomethyl)indol-4-ol
1-Ethyl-3-(ethylaminomethyl)indol-4-ol
1-Ethyl-3-[(propan-2-ylamino)methyl]indol-4-ol
3-(Ethylaminomethyl)-1-propan-2-ylindol-4-ol
1-Propan-2-yl-3-[(propan-2-ylamino)methyl]indol-4-ol
3-(2-Hydroxypropan-2-yl)-1H-indol-4-ol
3-(2-Hydroxy-2-methylpropyl)-1H-indol-4-ol
3-(Aminooxymethyl)-1H-indol-4-ol
1H-Indol-4-ol, 3-(1-aminoethyl)-
341-(Methylamino)ethyl]-1H-indol-4-ol
1-Methyl-3-[1-(methylamino)ethyl]indol-4-ol
3-[3-(Methylamino)propyl]-1 H-indol-4-ol
1-Methyl-3-[3-(methylamino)propyl]indol-4-ol
1-Ethyl-3-11-(methylaminotethyllIndol-4-ol
341-(Methylamincothyll-1-propan-2-ylindol-4-ol
1-Ethyl-342-(methylamino)propyl]indol-4-ol
3 2-Meth ′amino 1-n-2-lindol-4-ol
1-Ethyl-343-(methylamlno)propyllindol-4-ot
343-(Methylamino)ProPYIP-propan-2-ylindol-4-ol
544-Aminobutyl)-3-methyl-1H-indol-4-ol
1H-Indo1-4-ol, 3-propy1-
342-(Dimethylamino)-1-hydroxyethyl]-1H-indol-4-ol
3-I(3S)-1-Methylpyrrolidin-3-yl)-1H-indol-4-ol
34(3R)-1-Methylpyrrolidin-3-y11-111-indo1-4-ol
341-(Dimethylamino)propan-2-yll-1H-indol-4-ol
3-(2-AmInobutyl)-1 H-indol-4-ol
3 1-Amino-3-meth bu 1H-indol-4-ol
34(1S)-1-Amino-3-methylbuty11-1H-indol-4-ol
34(1 R)-1-Amino-3-methylbuty11-1H-indol-4-ol
3 1 Hro meth I amino eth-1H-Indol-4-ol
3-(N-Hydroxy-C-rnethylcarbonimidoyl)-11-1-indol-4-ol
[342-(13imethylazaniumygethyl]-1H-inclol-4-yl] hydrogen
phosphate; methanol
(3-(3-Ethylheptyl)-1H-indol-4-yl] hypolluorite
0-(3-Propy1-1H-indol-4-yl)hydroxylamine
(3-Pentan-3-14-1H-indol-4-0) hypofluortle
04342-(Methylamino)ethyll-1H-Indol-4-yllhydroxylamine
(3-Heptan-3-yl-1H-idol-4-y1) hypoffuorrte
N-Methyl-2-(4-sulfanyloxy-1H-indo1-3-yl)ethanamine
3-Ethyl-4-sulfanyloxy-1H-indole
N,N-Dimethyl-2-(4-suffanyloxy-1H-isiol-3-yqethanamine
0-13-(2-Aminoethyl)-1H-inciol-4-yllhydroxylamine
N-(1H-Indol-4-yloxy)-2-methylpropan-1-asnine
1H-Indo1-4-yl hydrogen sulfate
Psilocin iminoquinone
Psilocin o-quinone
O-acetyl psilocin fumarate
4-Acetoxyindole
4-Acetoxy-N,N-dimethyltryptamine
4-Acetoxy-N-isopropyl-N-methyltryptamine
4-Acetoxy-N-ethyl-N-methyltryptamine
4-Acetoxyindole
O-Acetyl Psilocin Fumarate
4-AcO-DET Fumarate
4-AcO-MET Fumarate
4-AcO-DET
O-Benzyl Psilocin
3-(2-(Dimethylamino)ethyl)-5,6,7,7a-tetrahydro-1 H-indol-4(3aH)-one
4-Hydroxy-N-isopropyl-N-methyltryptamine
4-Hydroxy McPT Hydrochloride
O-Methylpsilocin
1-Methylpsilocin
4-Methoxy MiPT Hydrochloride
(−)-Normacromerine
Psilocin O-Glucuronide

As each psilocybin analog can have different psychoactive effects when compared to another psilocybin analog, it will be recognized that a composition of the instant disclosure comprises intended one or more psilocybin analogs in a substantially pure form, absent other psylocibin analogs. Accordingly, a composition of the instant disclosure comprises no psylocibin analogs other than the one or more psilocybin analogs of the composition or comprises trace amounts of an unintended psilocybin analog at sufficiently low concentrations that do not exert non-therapeutic psychoactive effects on the subject.

Psilocybin is a naturally occurring psychedelic compound produced by more than 200 species of psychedelic fungi. The most potent are members of the genus Psilocybe, such as P. azurescens, P. semilanceata, and P. cyanescens. Psylocybins, analogs thereof, derivatives thereof, and psilocybin-like molecules are abundant in nature and can also be found in mushrooms from the genus Conocybe, Copelandia, Galerina, Gymnopilus, Inocybe, Panaeolus, Pholiotina, Pluteus as well as many fungi, plant species, bacteria, and animals, including mammals. For instance, bufotenine is a psilocybin-like compound found in the skin of psychoactive toads.

A psilocybin analog of the instant disclosure can be a naturally occurring psilocybin analog compound or a synthetic psilocybin analog that may not occur in nature. The psilocybin analog can be extracted and purified from a natural source or can be chemically synthesized. Methods of extracting and purifying psilocybin analogs are known in the art and can be as described in WO2021253116, the entire contents of which are incorporated herein in their entirety. Individuals of skill in the art will also be familiar with methods of chemical synthesis of a psilocybin analog compound.

Compositions of the instant disclosure can comprise one or more than one psilocybin analog. When a composition comprises more than one psilocybin analog, the activity of each psilocybin can be the same or different from the activity of another psilocybin analog in the composition. For instance, a composition of the instant disclosure can comprise a psilocybin analog having anxiolytic effects, and another psilocybin analog exhibiting anti-depressive activity. Alternatively, more than one psilocybin analog can have the same therapeutic psychoactive activity and exert an additive or synergistic therapeutic effect. Accordingly, a composition of the instant disclosure can comprise 1, 2, 3, 4, 5, 6, 7, 8, 9m, 10 or more psilocybin analogs.

Many psilocybin analogs present in natural sources can be in a phosphorylated form or a dephosphorylated form, although other non-phosphorylatable psychoactive alkaloids may also be present. In general, when the phosphorylated forms of the compounds are consumed (whether in the raw mushroom, purified extracts, or synthetic preparations of the compounds), the phosphorylated analogues are enzymatically metabolized by the human gut, liver, and kidneys to the active dephosphorylated forms. For instance, the phosphorylated pro-drug forms of psilocybin ([3-(2-Dimethylaminoethyl)-1H-indol-4-yl]phosphate), norbaeocystin, baeocystin and aeruginascin are metabolized to the active dephosphorylated forms psilocin, 4-hydroxytryptamine, norpsilocin and 4-hydroxy-N,N,N-trimethyltryptamine, respectively (Table 2). Accordingly, a psilocybin analog in a composition of the instant disclosure can be the phosphorylated pro-drug form or the active dephosphorylated form. The psilocybin analogs can also be in other pro-drug forms that can be used to improve how the drug is absorbed, distributed, metabolized, and excreted (ADME).

TABLE 2

| Phosphorylated form | Dephosphorylated metabolite |
|---|---|
| Psilocybin analog | Psilocin |
| 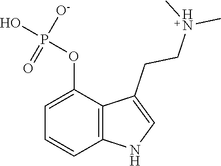 | 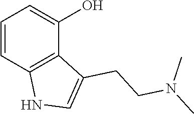 |
| Norbaeocystin | 4-hydroxytryptamine |
| 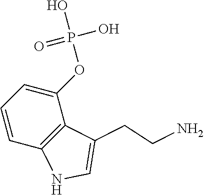 [SMILES: NCCc1c[nH]c2cccc(OP(=O)(O)O)c12] | 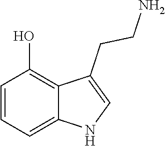 [SMILES: NCCc1c[nH]c2cccc(O)c12] |
| Baeocystin | Norpsilocin |
| 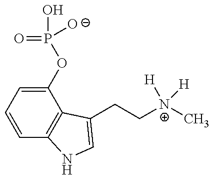 [SMILES: C[NH2+]CCc1c[nH]c2cccc(OP(=O)([O-])O)c12] | 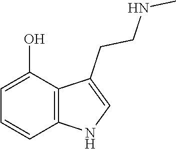 |
| Aeruginascin | 4-hydroxy-N,N,N-trimethyl- |
| | tryptamine |
| 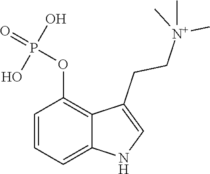 [SMILES: C[N+](C)(C)CCc1c[nH]c2cccc(OP(=O)(O)O)c12] | 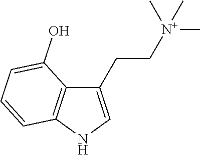 [SMILES: C[N+](C)(C)CCc1c[nH]c2cccc(O)c12] |

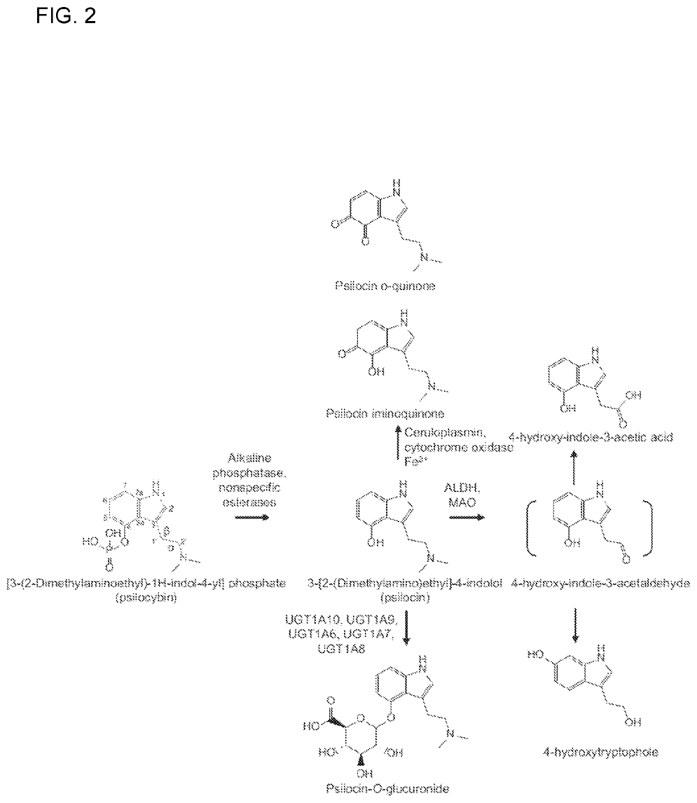

Psilocybin analogs can also be metabolized to generate other compounds that can have the same or different psychoactive activities or pharmacokinetic characteristics. A non-limiting example of the metabolism of a psilocybin analog is shown in FIG. 2. Since psilocin is structurally related to the neurotransmitter serotonin (FIG. 1 and FIG. 2), it undergoes comparable human metabolism. Indeed, psilocin is then further metabolized by a demethylation and oxidative deamination catalyzed by liver monoamine oxidase (MAO) or aldehyde dehydrogenase, via a presumed intermediate metabolite, 4-hydroxyindole-3-acetaldehyde, to yield 4-hydroxyindole-3-acetic acid, 4-hydroxy-indole-3-acetaldehyde and 4-hydroxytryptophole. It was also described a minor oxidation metabolic pathway of psilocin to a deep blue color product with an o-quinone or iminoquinone structure. This pathway was claimed to be catalyzed by hydroxyindol oxidases (e.g., ceruloplasmin, the copper containing oxidase of mammalian plasma and cytochrome oxidase) or non-enzymatically by Fe³⁺.

Monoamine oxidase inhibitors (MAOI) have been known to prolong and enhance the effects of DMT and one study assumed that the effect on psilocybin would be similar since it is a structural analogue of DMT. Alcohol consumption may enhance the effects of psilocybin, because acetaldehyde, one of the primary breakdown metabolites of consumed alcohol, reacts with biogenic amines present in the body to produce MAOIs related to tetrahydroisoquinoline and β-carboline. Tobacco smokers may also experience more powerful effects with psilocybin, because tobacco smoke exposure decreases the activity of MAO in the brain and peripheral organs.

In some aspects, the composition of the instant disclosure comprises baeocystin. In other aspects, the composition of the instant disclosure comprises norpsilocin. As explained herein above, baeocystin is the phosphorylated form of norpsilocin. The inventors surprisingly discovered that compositions comprising baeocystin or norpsilocin have anxiolytic activity and no harmful psychoactive effects, including no depressive activity. Baeocystin is a zwitterionic alkaloid and analog of psilocybin. It is found as a minor compound in most psilocybin mushrooms together with psilocybin, norbaeocystin, and psilocin. Baeocystin is an N-demethylated derivative of psilocybin. Reports on the hallucinogenic activities of baeocystin are conflicting.

### (b) Compositions

Compositions of the instant disclosure can be formulated in dosage unit formulations containing conventional nontoxic pharmaceutically acceptable adjuvants, excipients, and vehicles as desired. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

A composition of the instant disclosure can be formulated and administered to a subject by several different means. For instance, a composition may generally be administered parenterally, intraperitoneally, intravascularly, transdermally, subcutaneously, rectally, or intrapulmonarily in dosage unit formulations containing conventional nontoxic pharmaceutically acceptable adjuvants, carriers, excipients, and vehicles as desired.

In those aspects administered orally, compositions may be administered in any orally acceptable dosage form including, but not limited to a tablet, including a suspension tablet, a chewable tablet, an effervescent tablet or caplet; a pill; a powder such as a sterile packaged powder, a dispensable powder, or an effervescent powder; a capsule including both soft or hard gelatin capsules such as HPMC capsules; a lozenge; a sachet; a sprinkle; a reconstitutable powder or shake; a troche; pellets; granules; liquids; suspensions; emulsions; or semisolids and gels. Alternatively, the pharmaceutical compositions may be incorporated into a food product or powder for mixing with a liquid or administered orally after only mixing with a non-foodstuff liquid. Capsule and tablet formulations may include, but are not limited to binders, lubricants, and diluents. Aqueous suspension formulations may include but are not limited to dispersants, flavor-modifying agents, taste-masking agents, and coloring agents.

Compositions provided herein may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

Compositions provided herein may also be formulated as suppositories. In these embodiments, the composition may include a suitable non-irritating excipient that is solid at room temperature but liquid at rectal temperature and therefore will melt in the rectum to release the drug. Non-limiting examples of suitable excipients for rectal suppository embodiments include cocoa butter, beeswax, and polyethylene glycols.

Compositions provided herein may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane.

Compositions provided herein may also be formulated as transdermal formulations, for example as a suitable ointment, lotion, cream, pastes, medicated plaster, patch, or membrane that includes but is not limited to a compound, according to this disclosure, suspended or dissolved in one or more carriers. Non-limiting examples of suitable carriers for transdermal embodiments include mineral oil, liquid petrolatum, white petrolatum, propylene glycol, polyoxyethylene, polyoxypropylene compound, emulsifying wax, sorbitan monostearate, Polysorbate 60, cetyl esters wax, cetearyl alcohol, 2-octyldodecanol, benzyl alcohol and water. For these embodiments, the molecular weight of the composition may range from about 1 to about 50 Daltons.

Compositions provided herein may also be formulated for parenteral administration including, but not limited to, intravenously, by injection, or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions provided herein may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

### A. Excipients and Carriers

Formulations of various aspects may include the composition of the disclosure, along with an excipient and/or pharmaceutically acceptable carrier. Non-limiting examples of excipients include preservatives (antioxidants), flavor-modifying agents, coloring agents, chelating agents, antimicrobial agents, stabilizers, surfactants, tonicity agents such as NaCl, suspending agents, release-controlling polymers, and any combination thereof. As used herein, the term “pharmaceutically acceptable carrier” is intended to include any and all solvents, dispersion media, coatings, antibacterial and antifungal agents, isotonic and absorption delaying agents, and other excipients compatible with pharmaceutical administration. The use of such media and agents for pharmaceutically active substances is well known in the art. It will be recognized that carriers can provide more than one function in a composition. A more detailed description of carriers can be as described below.

i. Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apocarotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma-and delta-tocopherol), tocotrienols (i.e., alpha-, beta-, gamma-and delta-tocotrienols), tyrosol, vanillic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof.

ii. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

iii. Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include, but are not limited to, cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

iv. Colorants

Depending upon the aspect, it may be desirable to include a coloring agent. Suitable color additives include, but are not limited to, food, drug, and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects.

v. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

vi. Antimicrobial Agents

An antimicrobial agent may be included as an excipient to minimize the degradation of the compound according to this disclosure by microbial agents, including but not limited to, bacteria and fungi. Non-limiting examples of antimicrobials include parabens, chlorobutanol, phenol, calcium propionate, sodium nitrate, Na₂EDTA, and sulfites, including but not limited to sulfur dioxide, sodium bisulfite, and potassium hydrogen sulfite.

vii. Stabilizers

In some aspects, isotonifiers, sometimes known as “stabilizers”, are added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers such as polyvinylpyrrolidone monosaccharides, such as xylose, mannose, fructose, glucose; disaccharides such as lactose, maltose, sucrose and trisaccacharides such as raffinose; and polysaccharides such as dextran. In some aspects, the composition does not include stabilizers.

viii. Antimicrobial Agents

An antimicrobial agent may be included as an excipient to minimize the degradation of the compound according to this disclosure by microbial agents, including but not limited to, bacteria and fungi. Non-limiting examples of antimicrobials include para-bens, chlorobutanol, phenol, calcium propionate, sodium nitrate, Na₂EDTA, and sulfites, including but not limited to sulfur dioxide, sodium bisulfite, and potassium hydrogen sulfite.

ix. Stabilizers

In some aspects, isotonifiers, sometimes known as “stabilizers”, are added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers such as polyvinylpyrrolidone monosaccharides, such as xylose, mannose, fructose, glucose; disaccharides such as lactose, maltose, sucrose and trisaccacharides such as raffinose; and polysaccharides such as dextran.

x. Surfactants

Non-ionic surfactants or detergents (also known as “wetting agents”) can be used to help solubilize the therapeutic agent, as well as to protect the active ingredient against agitation-induced aggregation. Suitable non-ionic surfactants include polysorbates (20, 80, etc.), polyoxamers (184, 188, etc.), Pluronic polyols, and polyoxyethylene sorbitan monoethers (TWEEN®-20, TWEEN®-80, etc.).

### B. Encapsulation

A composition of the instant disclosure can comprise a psilocybin analog encapsulated in a drug vehicle. As used herein, the term “encapsulated” refers to an active ingredient such as a psilocybin analog of the instant disclosure encapsulated in a drug carrier or drug vehicle. A wide variety of drug delivery systems have been developed and studied, each of which has unique advantages and disadvantages. Non-limiting examples of drug vehicles include liposomes, polymeric micelles, microspheres, and nanoparticles among others. Different methods of attaching the drug to the carrier have been implemented, including adsorption, integration into the bulk structure, encapsulation, and covalent bonding. Different types of drug vehicles utilize different methods of attachment, and some vehicles can even implement a variety of attachment methods.

Non-limiting examples of nanoparticles include nano diamonds, nanofibers, protein-DNA complexes, protein-drug complexes, protein-drug conjugates, erythrocytes, virosomes, and dendrimers. Encapsulating drug compounds such as psilocybin analogs of the instant disclosure can provide numerous benefits such as increasing the stability and life of the compound being encapsulated, facilitate the manipulation of the product, and provide for the controlled release of the contents.

Polymeric micelles are drug vehicles formed by the aggregation of some amphiphilic molecules with an amphiphilic block copolymer. These vehicles form at some high concentration specific to the compounds used, called the critical micelle concentration. The addition of an amphiphilic block copolymer effectively lowers this critical micelle concentration by shifting the monomer exchange equilibrium.

Microspheres are hollow, micron-sized vehicles often formed via self-assembly of polymeric compounds which are most often used to encapsulate the active drug for delivery. Drug release from microspheres is often achieved by diffusion through pores in the microsphere structure or by degradation of the microsphere shell. Some assembly techniques, such as precision particle fabrication (PPF), can create microspheres capable of sustained control over drug release.

Liposomes are artificially spherical vesicles prepared from naturally derived phospholipid. They entail one or more lipid bilayers with discrete aqueous spaces. They are well established for a range of pharmaceutical and biomedical applications with the unique capability of entrapment of both hydrophilic (polar) and hydrophobic (nonpolar) compounds due to their amphipathic nature in aqueous media. For instance, hydrophobic compounds entrap in the bilayer membrane, while hydrophilic compounds encapsulate in the aqueous core. Liposomes serve as DDSs due to their versatile structure; biocompatibility; and the fact they are naturally nontoxic, non-immunogenic, and biodegradable. Liposomes have several advantages contributing to drug delivery. They have a role enhancing drug solubility, serving as a sustained release system, providing targeted drug delivery, reducing the toxic effect of drugs, providing protection against drug degradation, enhancing circulation half-life of APIs, being effective in overcoming multidrug resistance, improving the therapeutic index of the entrapped drug, and protecting APIs against their surrounding environment.

Numerous factors define liposomes properties such as the lipid composition, number of lipid bilayers, size, surface charge, and the method of preparation. Liposomal vesicles vary in size between 0.025 μm to 2.5 μm. They can be categorised according to the number of their layers (also referred to as lamellae): unilamellar (consisting of single phospholipid bilayer) or multilamellar (consisting of more than one unilamellar separated by layers of water (>500 nm)). Unilamellar vesicles are subdivided into small unilamellar vesicles (20-100 nm) and large unilamellar vesicles (>100 nm). Both the size and the number of lamellae in the liposomal structure are considered to be the most crucial factors affecting the vesicles half-life and the quantity of API that is to be encapsulated. This unique and flexible variety in the liposomal structure distinguishes liposomes as the preferred carriers for a broad spectrum of therapeutic agents.

Physical and chemical stability of the liposomes in terms of size distribution, entrapment efficiency, and minimal degradation of liposomal apparatuses is the major limiting step for drug delivery using this system. Chemical degradation of liposomes mainly occurs at the phospholipid bilayers level, in which two different reactions might develop: (i) hydrolysis of the ester bonds between fatty acids and glycerol backbone, and (ii) peroxidation of any available unsaturated acyl chain. These two reactions might lead to the development of short-chain lipids; subsequently, soluble derivatives will appear in the membrane that would significantly reduce the quality and stability of the liposomal system. With respect to physical instability, liposomes might undergo aggregation/flocculation and fusion/coalescence, which can ultimately change vesicle size and lead to significant loss of the encapsulated API.

Several factors that have an influence on liposomal system stability, such as liposomal composition (e.g., phospholipids-lipids with high phase transition temperatures), fatty acid side-chains, polar head chemistry, chain length, and the degree of unsaturation, can maintain liposomal rigidity and phospholipid:cholesterol molar ratio (crucial for the liposomal stability and controlling drug release).

Phospholipids are the main building blocks of liposomes. These biomolecules are also the main components building the biological membranes. They are amphiphilic molecules that consist of a polar head (water soluble hydroxy groups) and insoluble backbone. Liposomes can be zwitterionic, positively or negatively charged, or uncharged. This is dependent on the polar head charge. There are two types of lipids currently utilized for liposome preparation: naturally occurring or synthesized double-chain lipids (consisting of phosphorus polar head and glycerol backbone) and sterols (e.g., cholesterol).

The most known lipids used in the liposomal formulations are phosphatidylcholine (zwitterionic), phosphatidylglycerol (negatively charged), phosphatidic acid, phosphatidylethanolamine (zwitterionic), and phosphatidylserine (negatively charged). Positively charged lipids (e.g., N-[1-(2,3-dioleyloxy) propyl]-N,N,N-triethylammonium (DOTMA) and 1,2-dioleoyl-3-trimethylammoniopropane (DOTAP)) are mainly used for gene delivery, as they interact with the negatively charged deoxyribonucleic acid (DNA) and negatively charged APIs.

Cholesterol is another strategic component of liposomes. It has a modulatory effect on the properties of the lipid bilayer of the liposomes. It can control the stoutness in the liposome structure and increase the packing between the phospholipid molecules, resulting in more ordered conformation in the aliphatic tail region, reduced micropolarity, reduced bilayer flexibility to the surrounding molecules (especially water-soluble molecules), and increases in the microviscosity of the bilayer. Cholesterol is also crucial for structural stability of liposomal membranes against intestinal environmental stress. Cholesterol was found to influence liposomes size (increasing cholesterol concentration increases liposomes size in addition to shape transition), provide permeability and fluidity, and consequently modulate the release of hydrophilic compounds from liposomes.

The lipid bilayer of a liposome may fuse with other bilayers (e.g., the cell membrane), thus delivering the contents of the liposome to cells. Phospholipids generally comprise two fatty acids linked through glycerol phosphate to one of a variety of polar groups. Non-limiting examples of phospholids suitable for liposomes include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE), or any combination thereof.

The fatty acid chains may range from about 6 to about 26 carbon atoms in length, and the lipid chains may be saturated or unsaturated. Non-limiting examples of suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis, cis-9,12-octadecandienoate (linoleate), all-cis-9,12,15-octadecatrienoate (linolenate), and all-cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and any combination thereof.

The phospholipids can come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-diheptyloxacarbocyanine iodide, 1,1′-didodecyl-3,3,3′,3′-tetramethylindocarbocyanine perchloarate, 1,1′-dioleyl-3,3,3′,3′-tetramethylindo carbocyanine methanesulfonate, N,4-(dilinoleylaminostyryl)-N-methylpyridinium iodide, or 1,1-dilinoleyl-3,3,3′,3′-tetramethylindocarbocyanine perchloarate.

Liposomes can optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). The PEGylated lipids may generally increase the amount of compound that can be incorporated into the liposomes. PEGs may range in size from about 500 to about 10,000 Daltons. A suitable PEGylated phospholipid is dipalmitoyl PE bearing PEG 5,000.

Liposomes can further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the psilocybin analogs of the instant disclosure can be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, and Verrico et al., PAIN, 2020 Sep. 1; 161 (9): 2191-2202, the disclosures of which are hereby incorporated by reference in their entirety. For example, liposomes can be prepared by sonicating lipids in an aqueous solution, solvent injection, lipid hydration, reverse evaporation, or freeze drying by repeated freezing and thawing. The liposomes can be multilamellar, which have many layers like an onion, or unilamellar.

As would be apparent to one of ordinary skill, all the parameters that govern liposome formation may be varied. These parameters include, but are not limited to, temperature, pH, concentration of active pharmaceutical ingredient, concentration and composition of lipid, concentration of multivalent cations, rate of mixing, presence of and concentration of solvent.

### II. Method of Treating

Another aspect of the present disclosure encompasses a method of treating a psychological disorder in a subject in need thereof. The method comprises administering to the subject a therapeutically effective amount of a composition comprising a psilocybin analog. Importantly, using the compositions of the instant disclosure, methods of the instant disclosure can treat psychological disorders even when the psilocybin analog exhibits psychoactive activity in addition to the beneficial therapeutic activity of the psilocybin analog. Compositions comprising a psilocybin analog can be as described in Section I.

A composition of the instant disclosure can be administered to a subject by several different means. For instance, a composition may generally be administered parenterally, intraperitoneally, intravascularly, transdermally, subcutaneously, rectally, or intrapulmonarily in dosage unit formulations containing conventional nontoxic pharmaceutically acceptable adjuvants, carriers, excipients, and vehicles as desired.

The subject can be a human, a livestock animal, a companion animal, a lab animal, or a zoological animal. In one aspect, the subject may be a rodent, e.g., a mouse, a rat, a guinea pig, etc. Non-limiting examples of suitable livestock animals may include pigs, cows, horses, goats, sheep, llamas, and alpacas. Non-limiting examples of companion animals may include pets such as dogs, cats, rabbits, and birds. As used herein, a “zoological animal” refers to an animal that may be found in a zoo. Such animals may include non-human primates, large cats, wolves, and bears. Non-limiting examples of a laboratory animal may include rodents, canines, felines, and non-human primates. Non-limiting examples of rodents may include mice, rats, guinea pigs, etc.

In some aspects, the subject is a human subject. In some aspects, the disorder is coronary artery disease (narrowing of the blood vessels that supply blood to the heart).

### (a) Psychological Disorders

One aspect of the instant disclosure encompasses a method of treating a psychological disorder. The method comprises administering a therapeutically effective amount of a psilocybin analog to a subject in need thereof.

In some aspects, the method comprises administering baeocystin or norpsilocin. In some aspects, the method comprises administering baeocystin or norpsilocin at a therapeutically effective amount at sub-hallucinogenic concentrations. Little information exists with regard to human pharmacology, but in the book Magic Mushrooms Around the World, author Jochen Gartz reports being aware of a study in which “10 mg of baeocystin were found to be about as psychoactive as a similar amount of psilocybin.” Gartz also reported in a research paper that a self-administered assay of 4 mg of baeocystin caused “a gentle hallucinogenic experience”.

In some aspects, the method comprises administering a dose of baeocystin, norpsilocin, or a combination of baeocystin and norpsilocin of about 15, 14, 13, 12, 11, 10, 9, 8, 7, 6, 5, 4, 3, 2, or 1 mg or less. In some aspects, In some aspects, the method comprises administering a dose of baeocystin or norpsilocin of about, 10 mg or less. In some aspects, In some aspects, the method comprises administering a dose of baeocystin or norpsilocin of about, 4 mg or less.

In some aspects, the therapeutically effective amount of baeocystin, norpsilocin, or a combination of baeocystin and norpsilocin ranges from about 0.01 mg/kg to about 10 mg/kg body weight of the subject. In some aspects, the therapeutically effective amount of baeocystin, norpsilocin, or a combination of baeocystin and norpsilocin ranges from about 0.05 mg/kg to about 10 mg/kg, from about 0.1 mg/kg to about 10 mg/kg, from about 0.5 mg/kg to about 10 mg/kg, from about 1 mg/kg to about 10 mg/kg, from about 5 mg/kg to about 10 mg/kg, from about 0.01 mg/kg to about 9 mg/kg, from about 0. 01 mg/kg to about 8 mg/kg, from about 0.01 mg/kg to about 7 mg/kg, from about 0.01 mg/kg to about 6 mg/kg, from about 0.01 mg/kg to about 5 mg/kg, from about 0.01 mg/kg to about 4 mg/kg, from about 0.01 mg/kg to about 3 mg/kg, from about 0.01 mg/kg to about 2 mg/kg, from about 0.01 mg/kg to about 1 mg/kg, from about 0.01 mg/kg to about 0.5 mg/kg, from about 0.01 mg/kg to about 0.1 mg/kg, from about 0.1 mg/kg to about 10 mg/kg, from about 1 mg/kg to about 5 mg/kg, from about 2 mg/kg to about 4 mg/kg, or from about 2.5 mg/kg to about 3.5 mg/kg. In some aspects, the therapeutically effective amount of baeocystin, norpsilocin, or a combination of baeocystin and norpsilocin ranges from about 0.25 mg/kg to about 0.35 mg/kg. In some aspects, the therapeutically effective amount of baeocystin, norpsilocin, or a combination of baeocystin and norpsilocin ranges from about 2.5 mg/kg to about 3.5 mg/kg.

In some aspects, the therapeutically effective amount of baeocystin ranges from about 1 mg/kg to about 100 mg/kg body weight of the subject. In some aspects, the therapeutically effective amount of baeocystin, norpsilocin, or a combination of baeocystin and norpsilocin ranges from about 0.1 mg/kg to about 100 mg/kg, from about 0.5 mg/kg to about 100 mg/kg, from about 0.6 mg/kg to about 100 mg/kg, from about 0.7 mg/kg to about 100 mg/kg, from about 0.8 mg/kg to about 100 mg/kg, from about 0.9 mg/kg to about 100 mg/kg, from about 1 mg/kg to about 90 mg/kg, from about 1 mg/kg to about 80 mg/kg, from about 1 mg/kg to about 70 mg/kg, from about 1 mg/kg to about 60 mg/kg, from about 1 mg/kg to about 50 mg/kg, from about 1 mg/kg to about 40 mg/kg, from about 1 mg/kg to about 30 mg/kg, from about 1 mg/kg to about 20 mg/kg, from about 1 mg/kg to about 15 mg/kg, from about 2 mg/kg to about 90 mg/kg, from about 3 mg/kg to about 80 mg/kg, from about 4 mg/kg to about 70 mg/kg, from about 5 mg/kg to about 60 mg/kg, from about 6 mg/kg to about 50 mg/kg, from about 7 mg/kg to about 40 mg/kg, from about 8 mg/kg to about 30 mg/kg, from about 9 mg/kg to about 20 mg/kg, or from about 9.5 mg/kg to about 10.5 mg/kg. In some aspects, the therapeutically effective amount of baeocystin ranges from about 9.5 mg/kg to about 10.5 mg/kg body weight of the subject.

In some aspects, the therapeutically effective amount of baeocystin ranges from about 0.1 mg/kg to about 100 mg/kg body weight of the subject. In some aspects, the therapeutically effective amount of baeocystin, norpsilocin, or a combination of baeocystin and norpsilocin ranges from about 0.5 mg/kg to about 100 mg/kg, from about 1 mg/kg to about 100 mg/kg, from about 5 mg/kg to about 100 mg/kg, from about 10 mg/kg to about 100 mg/kg, from about 15 mg/kg to about 100 mg/kg, from about 20 mg/kg to about 100 mg/kg, from about 25 mg/kg to about 100 mg/kg, from about 0.1 mg/kg to about 95 mg/kg, from about 0. 1 mg/kg to about 90 mg/kg, from about 0.1 mg/kg to about 85 mg/kg, from about 0.1 mg/kg to about 80 mg/kg, from about 0.1 mg/kg to about 75 mg/kg, from about 0.1 mg/kg to about 70 mg/kg, from about 0.1 mg/kg to about 65 mg/kg, from about 0.1 mg/kg to about 60 mg/kg, from about 0.1 mg/kg to about 50 mg/kg, from about 0.1 mg/kg to about 45 mg/kg, from about 0.1 mg/kg to about 40 mg/kg, from about 0.1 mg/kg to about 35 mg/kg, from about 1 mg/kg to about 90 mg/kg, from about 10 mg/kg to about 80 mg/kg, from about 15 mg/kg to about 70 mg/kg, from about 20 mg/kg to about 60 mg/kg, from about 25 mg/kg to about 50 mg/kg, from about 25 mg/kg to about 40 mg/kg, or from about 25 mg/kg to about 35 mg/kg. In some aspects, the therapeutically effective amount of baeocystin ranges from about 25 mg/kg to about 35 mg/kg.

In some aspects, the therapeutically effective amount of baeocystin ranges from about 0.1 mg/kg to about 100 mg/kg body weight of the subject. In some aspects, the therapeutically effective amount of baeocystin, norpsilocin, or a combination of baeocystin and norpsilocin ranges from about 0.5 mg/kg to about 100 mg/kg, from about 1 mg/kg to about 100 mg/kg, from about 5 mg/kg to about 100 mg/kg, from about 10 mg/kg to about 100 mg/kg, from about 15 mg/kg to about 100 mg/kg, from about 20 mg/kg to about 100 mg/kg, from about 25 mg/kg to about 100 mg/kg, from about 30 mg/kg to about 100 mg/kg, from about 35 mg/kg to about 100 mg/kg, from about 40 mg/kg to about 100 mg/kg, from about 45 mg/kg to about 100 mg/kg, from about 0.1 mg/kg to about 95 mg/kg, from about 0. 1 mg/kg to about 90 mg/kg, from about 0.1 mg/kg to about 85 mg/kg, from about 0.1 mg/kg to about 80 mg/kg, from about 0.1 mg/kg to about 75 mg/kg, from about 0.1 mg/kg to about 70 mg/kg, from about 0.1 mg/kg to about 65 mg/kg, from about 0.1 mg/kg to about 60 mg/kg, from about 0.1 mg/kg to about 65 mg/kg, from about 5 mg/kg to about 95 mg/kg, from about 10 mg/kg to about 90 mg/kg, from about 15 mg/kg to about 85 mg/kg, from about 20 mg/kg to about 80 mg/kg, from about 25 mg/kg to about 75 mg/kg, from about 30 mg/kg to about 70 mg/kg, from about 35 mg/kg to about 65 mg/kg, from about 40 mg/kg to about 60 mg/kg, or from about 45 mg/kg to about 55 mg/kg. In some aspects, the therapeutically effective amount of baeocystin ranges from about 45 mg/kg to about 55 mg/kg.

In some aspects, the therapeutically effective amount of baeocystin ranges from about 10 mg/kg to about 300 mg/kg body weight of the subject. In some aspects, the therapeutically effective amount of baeocystin, norpsilocin, or a combination of baeocystin and norpsilocin ranges from about 20 mg/kg to about 300 mg/kg, from about 30 mg/kg to about 300 mg/kg, from about 40 mg/kg to about 300 mg/kg, from about 50 mg/kg to about 300 mg/kg, from about 60 mg/kg to about 300 mg/kg, from about 70 mg/kg to about 300 mg/kg, from about 80 mg/kg to about 300 mg/kg, from about 90 mg/kg to about 300 mg/kg, from about 100 mg/kg to about 300 mg/kg, from about 110 mg/kg to about 300 mg/kg, from about 120 mg/kg to about 300 mg/kg, from about 130 mg/kg to about 300 mg/kg, from about 140 mg/kg to about 300 mg/kg, from about 150 mg/kg to about 300 mg/kg, about 160 mg/kg to about 300 mg/kg, from about 165 mg/kg to about 300 mg/kg from about 10 mg/kg to about 290 mg/kg, from about 10 mg/kg to about 280 mg/kg, from about 10 mg/kg to about 270 mg/kg, from about 10 mg/kg to about 260 mg/kg, from about 10 mg/kg to about 250 mg/kg, from about 10 mg/kg to about 240 mg/kg, from about 10 mg/kg to about 230 mg/kg, from about 10 mg/kg to about 220 mg/kg, from about 10 mg/kg to about 210 mg/kg, from about 10 mg/kg to about 200 mg/kg, from about 10 mg/kg to about 190 mg/kg, from about 10 mg/kg to about 180 mg/kg, from about 10 mg/kg to about 185 mg/kg, from about 20 mg/kg to about 280 mg/kg, from about 30 mg/kg to about 270 mg/kg, from about 40 mg/kg to about 260 mg/kg, from about 50 mg/kg to about 250 mg/kg, from about 60 mg/kg to about 240 mg/kg, from about 70 mg/kg to about 230 mg/kg, from about 80 mg/kg to about 220 mg/kg, from about 90 mg/kg to about 210 mg/kg, from about 100 mg/kg to about 200 mg/kg, from about 110 mg/kg to about 190 mg/kg, from about 130 mg/kg to about 180 mg/kg, from about 140 mg/kg to about 180 mg/kg, from about 150 mg/kg to about 180 mg/kg, from about 160 mg/kg to about 180 mg/kg, or from about 165 mg/kg to about 175 mg/kg. In some aspects, the therapeutically effective amount of baeocystin ranges from about 165 mg/kg to about 175 mg/kg.

In some aspects, the therapeutically effective amount of baeocystin, norpsilocin, or a combination of baeocystin and norpsilocin ranges from about 100 mg/kg to about 300 mg/kg body weight of the subject. In some aspects, the therapeutically effective amount of baeocystin, norpsilocin, or a combination of baeocystin and norpsilocin ranges from about 110 mg/kg to about 300 mg/kg, 120 mg/kg to about 300 mg/kg, 130 mg/kg to about 300 mg/kg, 140 mg/kg to about 300 mg/kg, 150 mg/kg to about 300 mg/kg, 160 mg/kg to about 300 mg/kg, 100 mg/kg to about 290 mg/kg, 100 mg/kg to about 280 mg/kg, 100 mg/kg to about 270 mg/kg, 100 mg/kg to about 260 mg/kg, 100 mg/kg to about 250 mg/kg, 100 mg/kg to about 240 mg/kg, 100 mg/kg to about 230 mg/kg, 100 mg/kg to about 220 mg/kg, 100 mg/kg to about 1 mg/kg, 100 mg/kg to about 0.5 mg/kg, 100 mg/kg to about 0.1 mg/kg, 0.1 mg/kg to about 5 mg/kg, 0.2 mg/kg to about 4 mg/kg, 0.3 mg/kg to about 3 mg/kg. In some aspects, the therapeutically effective amount of baeocystin, norpsilocin, or a combination of baeocystin and norpsilocin ranges from about 0.3 mg/kg to about 3 mg/kg.

In some aspects, the therapeutically effective amount of baeocystin ranges from about 0.01 mg/kg to about 10 mg/kg body weight of the subject. In some aspects, the therapeutically effective amount of baeocystin, norpsilocin, or a combination of baeocystin and norpsilocin ranges from about 0.05 mg/kg to about 10 mg/kg, 0.1 mg/kg to about 10 mg/kg, 0.5 mg/kg to about 10 mg/kg, 1 mg/kg to about 10 mg/kg, 5 mg/kg to about 10 mg/kg, 0.01 mg/kg to about 9 mg/kg, 0. 01 mg/kg to about 8 mg/kg, 0.01 mg/kg to about 7 mg/kg, 0.01 mg/kg to about 6 mg/kg, 0.01 mg/kg to about 5 mg/kg, 0.01 mg/kg to about 4 mg/kg, 0.01 mg/kg to about 3 mg/kg, 0.01 mg/kg to about 2 mg/kg, 0.01 mg/kg to about 1 mg/kg, 0.01 mg/kg to about 0.5 mg/kg, 0.01 mg/kg to about 0.1 mg/kg, 0.1 mg/kg to about 5 mg/kg, 0.2 mg/kg to about 4 mg/kg, 0.3 mg/kg to about 3 mg/kg. In some aspects, the therapeutically effective amount of baeocystin, norpsilocin, or a combination of baeocystin and norpsilocin ranges from about 0.3 mg/kg to about 3 mg/kg.

The term psychological disorders is sometimes used to refer to what are more frequently known as mental disorders or psychiatric disorders. Mental disorders are patterns of behavioral or psychological symptoms that impact multiple areas of life. These disorders create distress for the person experiencing the symptoms. Non-limiting examples of psychological disorders include anxiety disorder, bipolar disorder, dementia, ADHD, schizophrenia, OCD, Autism, PTSD, addiction/substance abuse, various forms of depression, post-traumatic stress disorder, more commonly known as PTSD, schizophrenia, and suicidal thoughts. In some aspects, the method of the instant disclosure comprises administering a therapeutically effective amount of a psilocybin analog to treat depression. In some aspects, the method of the instant disclosure comprises administering a therapeutically effective amount of a psilocybin analog to treat anxiety. In some aspects, the method of the instant disclosure comprises administering a therapeutically effective amount of baeocystin, norpsilocin, or a combination of baeocystin and norpsilocin to treat depression. In some aspects, the method of the instant disclosure comprises administering baeocystin, norpsilocin, or a combination of baeocystin and norpsilocin to treat depression at a therapeutically effective amount ranging from about 0.3 mg/kg to about 3 mg/kg.

### (b) Administration

A composition of the instant disclosure can be administered to a subject by several different means. For instance, a composition can generally be administered parenterally, buccally, nasally, by inhalation, intraperitoneally, intravascularly, transdermally, subcutaneously, rectally, or intrapulmonarily. The term parenteral as used herein includes subcutaneous, intravenous, intramuscular, intrathecal, or intrasternal injection, or infusion techniques. Topical administration may also involve the use of transdermal administration such as transdermal patches or iontophoresis devices. The compositions can be formulated in dosage unit formulations for administration comprising conventional nontoxic pharmaceutically acceptable adjuvants, carriers, excipients, and vehicles as described in Section I(b)(B)).

Actual dosage levels of active ingredients in a therapeutic composition of the disclosure may be varied so as to administer an amount of a psilocybin analog that is effective to achieve the desired therapeutic response for a particular subject. A selected dosage level may depend upon a variety of factors, including the psilocybin analog in a composition, the activity of the therapeutic composition, formulation, the combination with other drugs or treatments, disease and longevity, the inflammation or inflammatory disorder, and the physical condition and prior medical history of the subject being treated. Determination of the proper dosage for a particular situation is within the skill of the practitioner.

In some aspects, the composition is administered regularly by following a prescribed treatment schedule. For instance, a composition of the instant disclosure can be administered routinely, at various intervals. For instance, compositions can be administered daily, weekly, monthly, or over several months. In some aspects, compositions are administered daily. In other aspects, compositions are administered weekly. In yet other aspects, compositions are administered monthly. Compositions can also be administered every three to six months. As it will be recognized in the art, the duration of treatment can and will vary and can be determined experimentally.

Administration of the compositions described herein can also be carried out as part of a treatment regimen that may include multiple instances of administration of one or more compositions comprising psilocybin analogs. Such a regimen may be designed as a method of immediately treating a condition and/or as a method of long-term maintenance of the health of a subject after having been treated for a condition (e.g., prevention). For instance, a treatment regimen can be designed to delay the onset of the condition of interest in a subject. It will be appreciated that determination of appropriate treatment regimens is within the skill of practitioners in the art.

It will also be appreciated by those skilled in the art that a composition of the present disclosure may be co-administered with other therapeutic agents before, after, and/or during treatment with a composition of the disclosure. The term “co-administer” refers to administration of more than one active ingredient at the same time, just prior to, or just after the administration of one or more additional therapies. The compounds of the disclosure can be administered alone or can be co-administered to the patient. Co-administration is meant to include simultaneous or sequential administration of the compounds individually or in combination. Dosage amounts and intervals can be adjusted individually to provide levels of the administered compound effective for the particular clinical indication being treated. This will provide a therapeutic regimen that is commensurate with the severity of the individual's disease state.

### III. Kits

A further aspect of the present disclosure provides kits comprising one or more pharmaceutical compositions comprising a psilocybin analog detailed above in Section II. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall beinterpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “oneof,” “only one of,” or “exactly one of.”

As used herein, the terms “including”, “includes”, “having”, “has”, “with”, or variants thereof, are intended to be inclusive similar to the terms “comprising” and “comprises.”

The term “therapeutically effective amount” as used with reference to the present formulation(s) and/or component(s) thereof as described herein refers to the quantity of the formulation(s) and/or component(s) thereof necessary to render the desired therapeutic result. For example, an effective amount is a level effective to treat, cure, or alleviate the symptoms of adisorder for which the therapeutic formulation is being administered. Amounts effective for the particular therapeutic goal sought will depend upon a variety of factors including: the disorder being treated and its severity and/or stage of development/progression; the bioavailability and activity of the specific compound, biologic or pharmaceutical composition used; the route or method of administration and introduction site on the subject; the rate of clearance of the specificcompound or biologic and other pharmacokinetic properties; the duration of treatment; inoculation regimen; drugs used in combination or coincident with the specific compound, biologic or composition; the age, body weight, sex, diet, physiology and general health of the subject being treated; and, like factors well known to one of skill in the relevant art. Some variation in dosage will necessarily occur depending upon the condition of the subject being treated, and the physician or other individual administering treatment will, in any event, determine the appropriate dosage for each individual patient.

As used herein, the term “administering” refers to providing a therapeutically effective amount of a formulation and/or components thereof to a patient via any of a number of potential delivery mechanisms including, but not limited to, oral, intravenous, transdermal, topical and/or inhalation, and the like. The formulation and/or components thereof of the present invention can be administered individually, but may also be administered with other compounds, excipients, fillers, binders, carriers or other vehicles selected based upon the chosen route of administration and standard pharmaceutical practice.

As used herein, the term “disorder” refers to a disorder, disease, condition, or other departure from healthy or normal biological activity, and can be used interchangeably. The condition may be caused by any of a number of physical factors. The condition may be caused by sporadic or inheritable genetic abnormalities. The condition may also be caused by non-genetic abnormalities. The condition may also be caused by injuries to a subject from environmental factors.

As used herein, the terms “treatment” or “treating” refers to arresting, inhibiting, correcting, or attempting to arrest or inhibit or correct, the existence, development, or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms.

As used herein, the term “treating” refers to: (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of a neurodegenerative disorder, “treat” and “treating” encompass alleviating, ameliorating, delaying the onset of, inhibiting the progression of, or reducing the severity of one or more symptoms associated with the neurodegenerative disorder.

“Acute inflammatory conditions” as the term is used herein, and in accordance with normal medical parlance, refers to inflammatory conditions having a rapid onset and severe symptoms. The duration of the onset, from a normal condition of the patient to one in which symptoms of inflammation are seriously manifested, is anything up to about 72 hours. Acute inflammatory conditions are to be contrasted with chronic inflammatory conditions, which are inflammatory conditions of long duration, denoting a disease showing little change or of slow progression. The distinction between acute and chronic conditions is well known to those in the medical professions, even if they are not distinguishable by rigid, numbers-based definitions.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1

### The Impact of Baeocystin, Norbacocystin, Aeruginascin and Norpsilocin in Animal Models of Anxiety and Depression in Mice

Summary. The goal of the present project was to assess psilocybin metabolites in animal models of anxiety and depression in mice. Specifically, baeocystin (0.3 mg/kg-30 mg/kg), norbaeocystin (0.3-3.0 mg/kg), aeruginascin (0.3-3.0 mg/kg) and norpsilocin (0.3-3.0 mg/kg) were tested in the open field test (OFT), elevated plus maze (EPM), tail suspension test (TST) and forced swim test (FST). Overall, most compounds were ineffective and/or increased negative behaviors. However, baeocystin (30 mg/kg) in the EPM significantly increased time on the open arm and norpsilocin significantly increased time spent in the center zone of the OFT. Both of these results suggest that baeocystin and norpsilocin can exert anxiolytic effects in mice using certain behavioral procedures.

Methods. The open field test (OFT) was conducted by placing a mouse in a gray chamber 40×40 cm of 30 cm height and allowed to explore at will for 30 minutes. A digital camera positioned above the chamber recorded behavior. Animals that spend more time in the center (open area) compared to along the walls (thigmotaxis) are deemed less anxious. The open field apparatus was cleaned with Peroxigard wipes after each use.

The elevated plus maze (EPM) is a behavioral assay that measures anxiety in rodents. The apparatus is composed of open and closed arms arranged in a “plus” measuring, 35 cm arm length; 5 cm arm width; 20 cm wall height and stands 61 cm height. Each mouse was placed on the maze within the center area facing towards an open arm and allowed to explore the EPM for 5 minutes. The experimenter observed the mouse from outside the test room through an observational window. Behavior was recorded via a digital camera mounted above the maze. The apparatus was cleaned with Peroxigard wipes after each trial.

The tail-suspension test (TST) is a model of depression and has been used to screen prospective antidepressant compounds. It involves suspending mice above the ground by their tails with tape for a predetermined time. A 17 cm piece of tape was taped to the tail (2 cm of the 17 cm) of each mouse and then attached to a metal shelf for 6 minutes. Each mouse was digitally recorded and data analyzed using specialized software with particular focus on time immobile. After the trial, tape was removed and mice returned to their home cage.

The forced swim test is a classic behavioral assay to test for depressive behavior such as immobility. The apparatus is composed of a Plexiglas cylinder measuring (20 cm diameter, 45 cm height) that is filled with water maintained at room temperature (23-25 C). During testing, mice were placed into the water by the nape of the neck facing the wall and released and behavior recorded via a digital camera for 6 minutes. After the test, mice were dried off with a towel and placed in their home cage in which half is heated by a heating pad placed underneath the cage. Fecal matter was removed after every test. Water was changed after 10 mice were run in each cylinder. After the completion of the experiment, water was drained and the cylinder cleaned with Peroxigard wipes.

Statistical analysis. Data were analyzed using a one-way ANOVA with drug dose as the primary factor. Post-hoc multiple comparison analysis was performed following significant main effects using Tukey's multiple comparisons test (GraphPad Prism 9.2.0). Statistical significance was set at P<0.05.

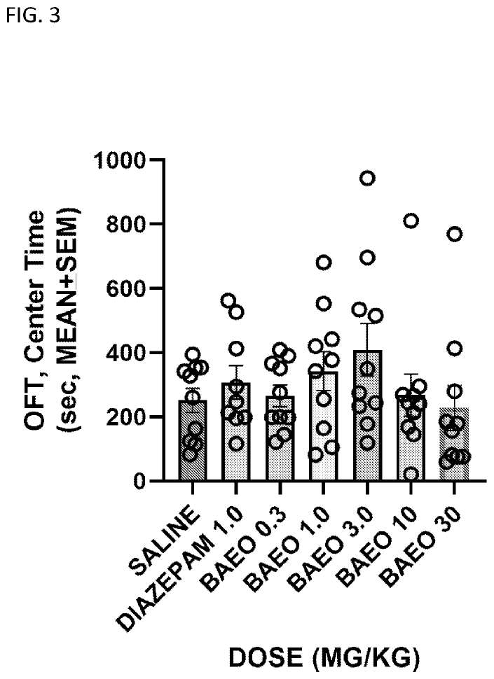

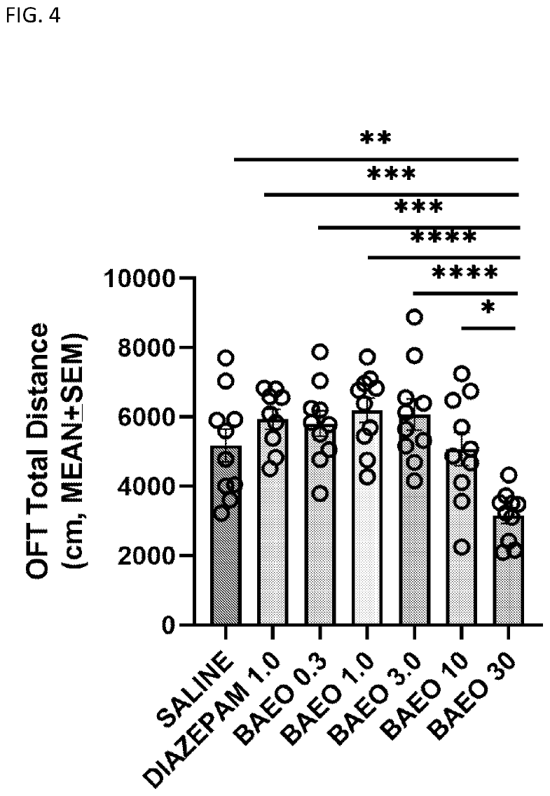

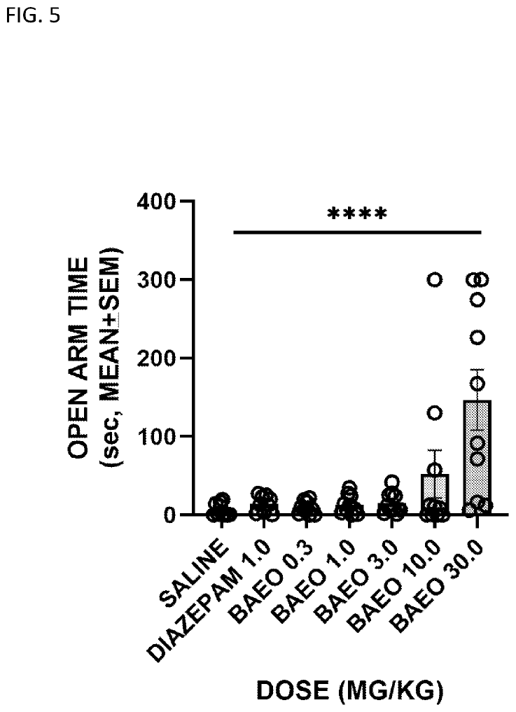

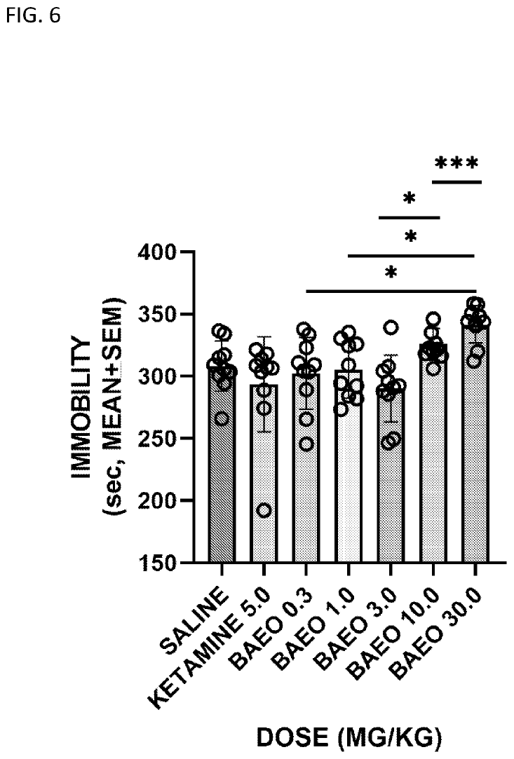

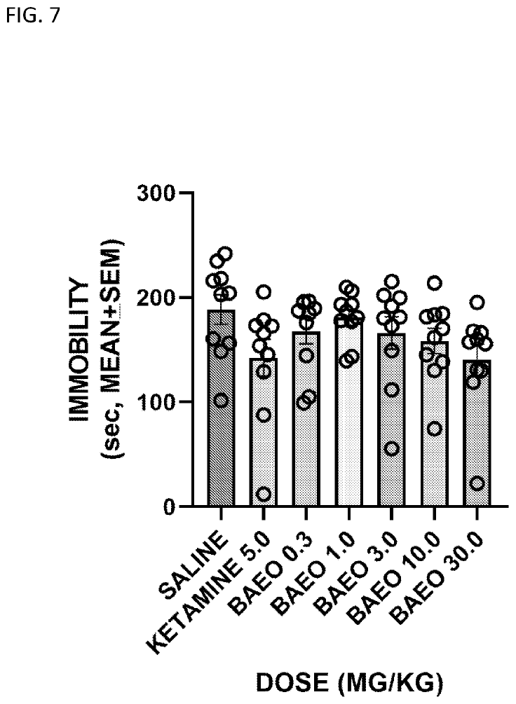

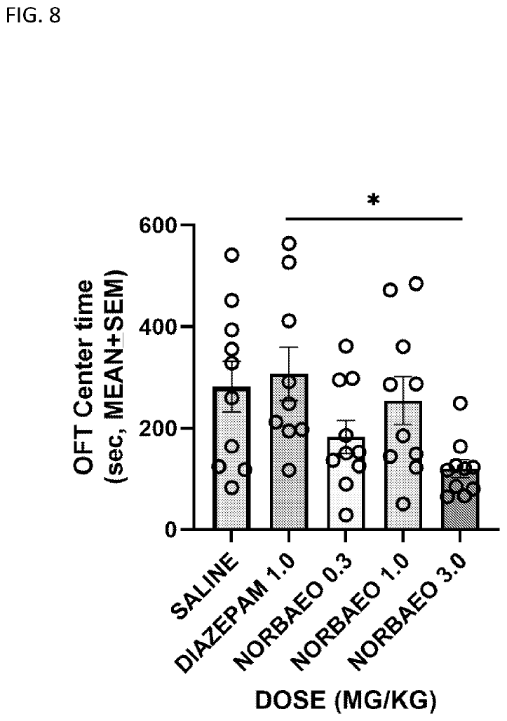

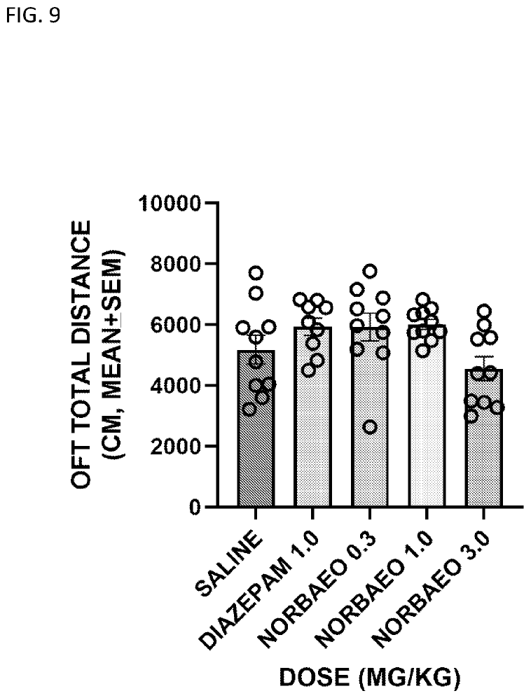

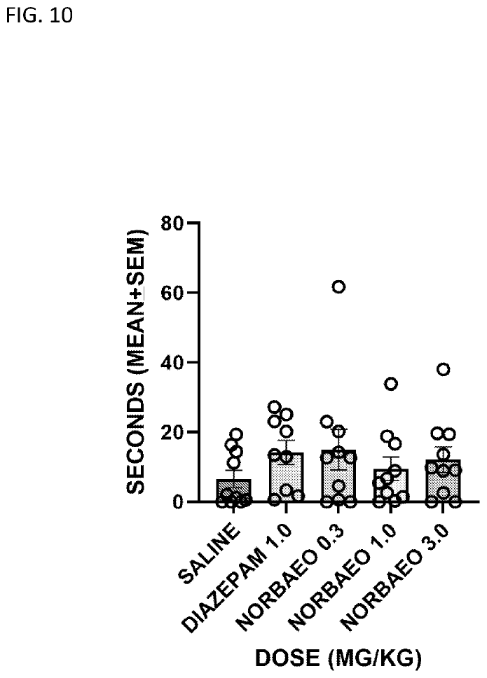

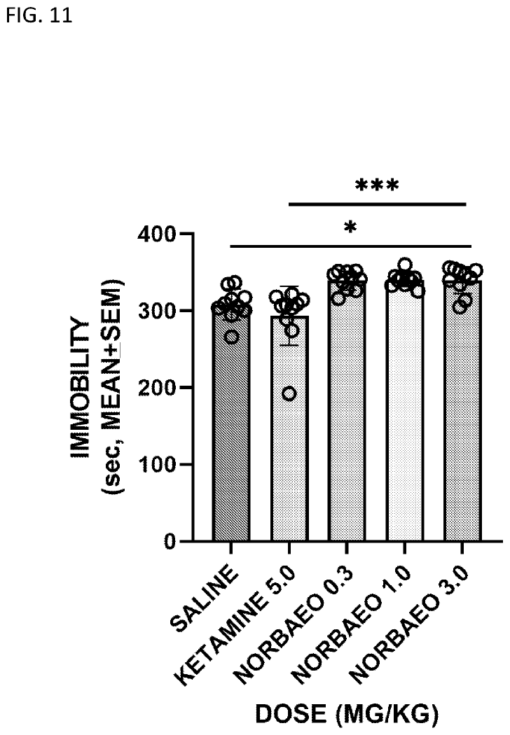

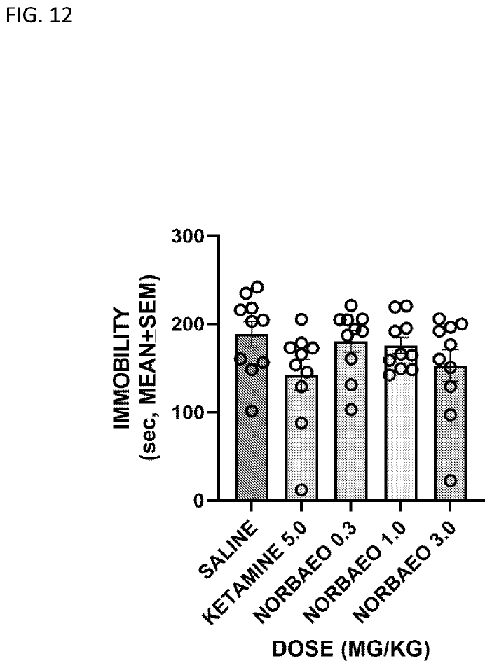

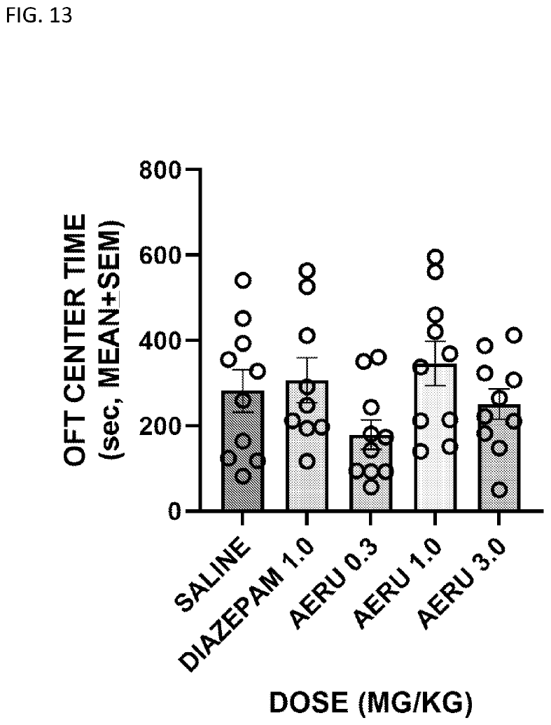

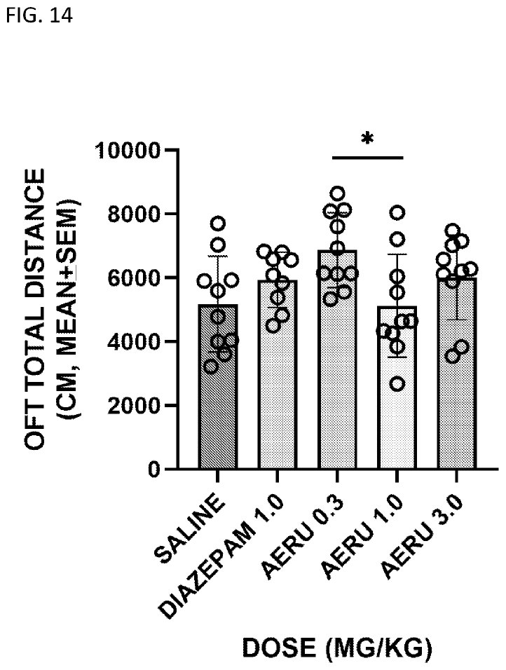

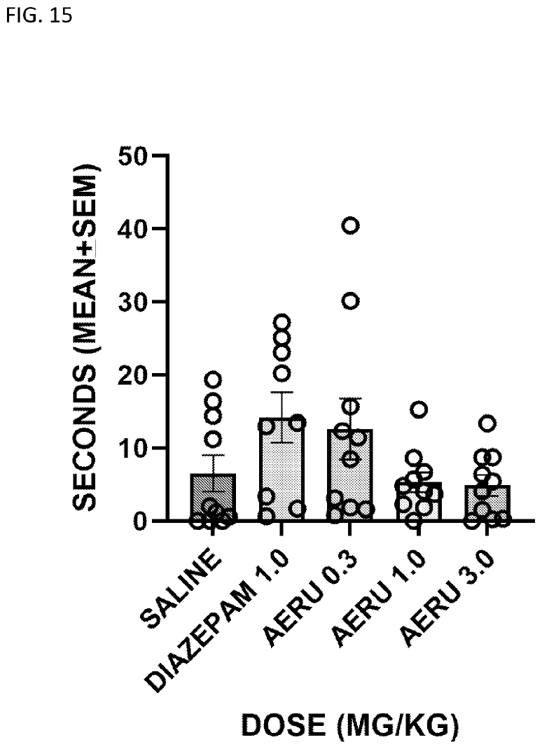

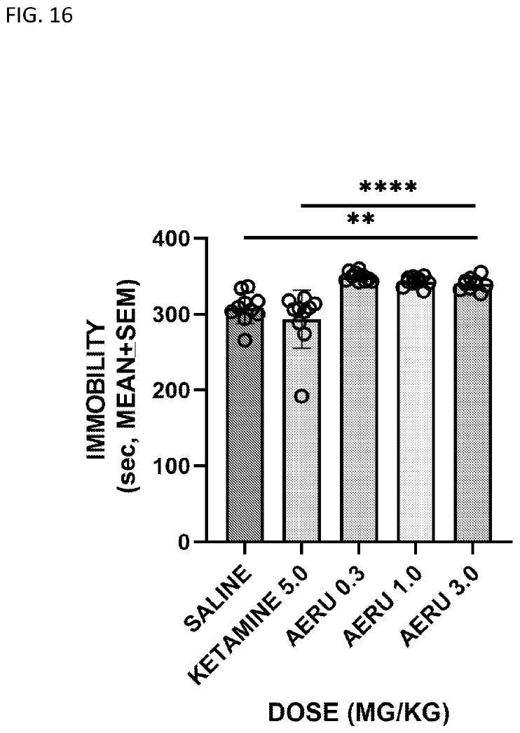

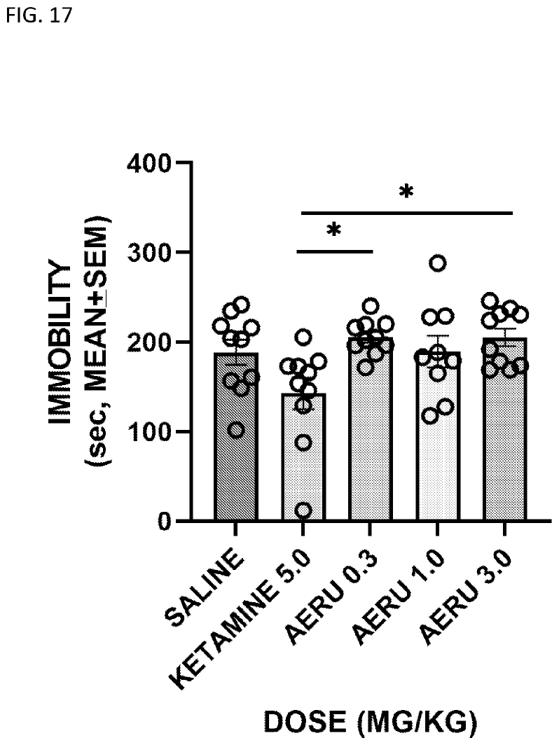

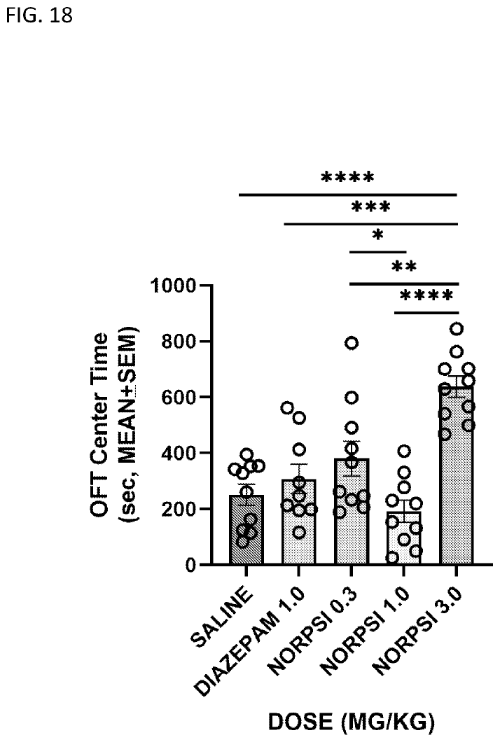

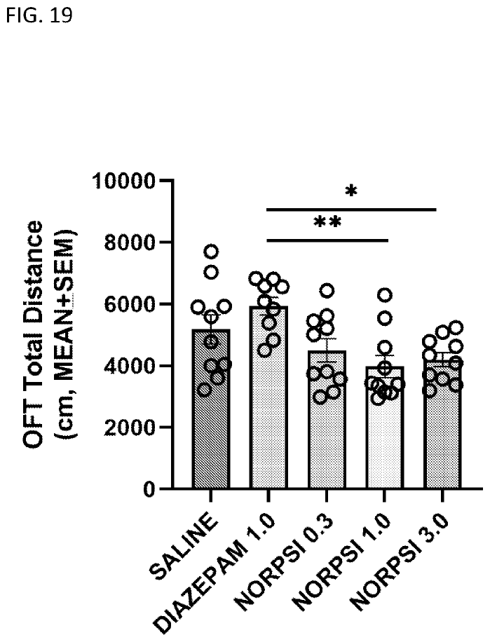

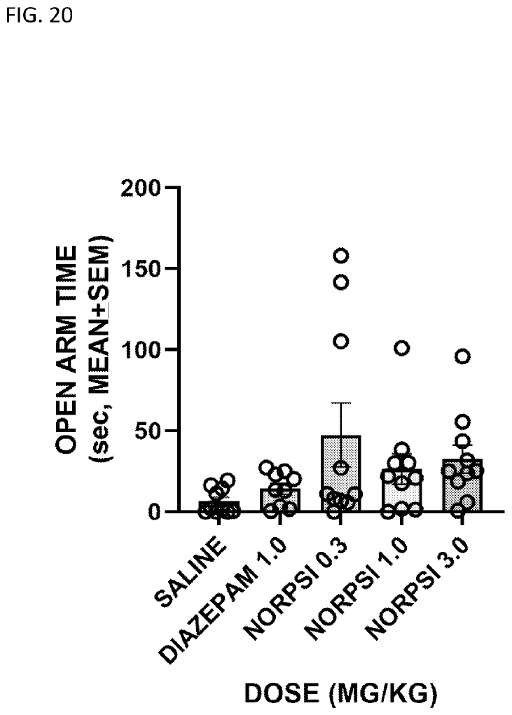

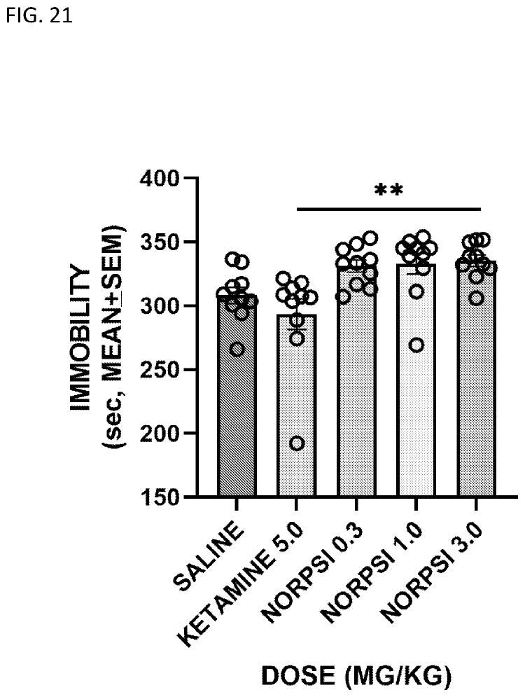

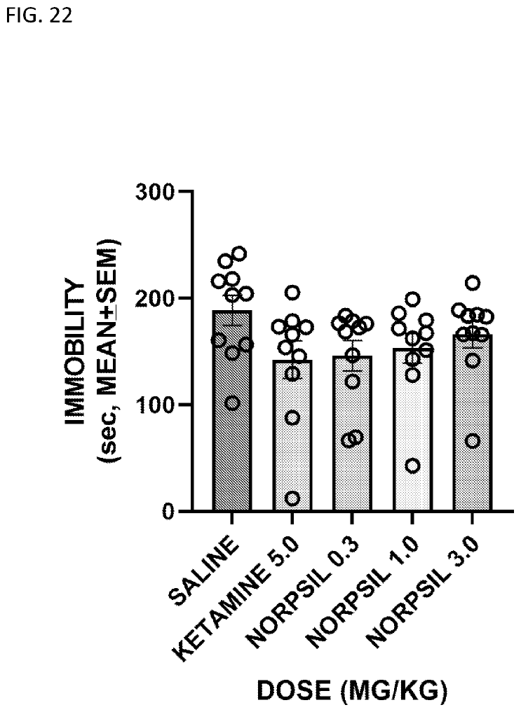

Results. A total of 170 male C57BL/6 mice were used for this study (see TABLE 3.). One mouse in the diazepam group died of unknown causes. Another mouse developed a skin condition in the aeruginascin (1.0 mg/kg) group and was excluded from the FST test. All other test groups consisted of N=10 per group. Statistical comparisons are noted in each figure legend. The quantitative data is shown in FIGS. 2-22.

TABLE 3
Treatment groups

| Drug | N |
|---|---|
| Saline | 10 |
| Diazepam (positive control, EPM, OFT) | 10 |
| Ketamine (positive control, FST, TST) | 10 |
| Baeocystin 0.3 mg/kg, IP | 10 |
| Baeocystin 1.0 mg/kg, IP | 10 |
| Baeocystin 3.0 mg/kg, IP | 10 |
| Baeocystin 10.0 mg/kg, IP | 10 |
| Baeocystin 30.0 mg/kg, IP | 10 |
| Norbaeocystin 0.3 mg/kg, IP | 10 |
| Norbaeocystin 1.0 mg/kg, IP | 10 |
| Norbaeocystin 3.0 mg/kg, IP | 10 |
| Aeruginascin 0.3 mg/kg, IP | 10 |
| Aeruginascin 1.0 mg/kg, IP | 10 |
| Aeruginascin 3.0 mg/kg, IP | 10 |
| Norpsilocin 0.3 mg/kg, IP | 10 |
| Norpsilocin 0.3 mg/kg, IP | 10 |
| Norpsilocin 0.3 mg/kg, IP | 10 |
| TOTAL | 170 |

* Baeocystin dose Sherwood et al. 2020

Interpretation and conclusions. TABLE 4 summarizes the effects of the various compounds tested in the aforementioned behavioral paradigms. Results indicate, norbaeocystin and aeruginascin either produced no significant effect or a significant negative impact across all behavioral tests. None of the compounds tested showed any positive effects in the two models of depression (TST, FST). The OFT in general assesses overall gross motor activity of an animal. All compounds but norbaeocystin significantly decreased activity. A decrease in overall activity must be considered a potential confound within the context of other positive behavioral effects. Norpsilocin (3.0 mg/kg) was the only compound that increased time spent in the center zone in the OFT and this effect was highly significant. Increased time spent in the center zone is generally interpreted as an anti-anxiety effect. Further, the highest dose of baeocystin (30 mg/kg) significantly increased time spent on the open arm in the EPM assay, which is also interpreted as an anti-anxiety effect. This anti-anxiety effect appeared to be dose-responsive in that the next lower dose of baeocystin (10 mg/kg) also tended to increase time spent on the open arms however, this did not reach statistical significance. The EPM is a classic behavioral paradigm that has strong predictive validity. That is, compounds producing significant anti-anxiety effects in the EPM show similar effects in humans. Doses of baeocystin and norpsilocin tested translate to 170.1 mg and 17.0 mg/day respectively for a 70 kg human and are within the scope of clinical application. This is the first series of studies to assess these compounds in animal models of anxiety and depression thus comparisons to previous studies is not possible.

TABLE 2
Summary of the effects of metabolites on behavior

| | OFT (center | | | | |
|---|---|---|---|---|---|
| | time) | OFT | EPM | TST | FST |
| Baeocystin | ns | ↓ (neg) | ↑ (pos) | ↑ (neg) | ns |
| Norbaeocystin | ↓ (neg) | ns | ns | ↑ (neg) | ns |
| Aeruginascin | ns | ↓ (neg) | ns | ↑ (neg) | ↑ (neg) |
| Norpsilocin | ↑ (pos) | ↓ (neg) | ns | ↑ (neg) | ns |

↓ = decrease, ↑ = increase, neg = negative, pos = positive, ns = not statistically significant, OFT = Open Field Test, EPM = Elevated Plus Maze, TST = tail suspension test, FST = Forced Swim Test.

1. A method of treating a psychological disorder in a subject in need thereof, the method comprising administering to the subject a therapeutically effective amount of a composition comprising a psilocybin analog.

2. The method of claim 1, wherein administering the composition comprises administering a unit dose of the composition to the subject, wherein the unit dose comprises a therapeutically effective amount of the psilocybin analog.

3. The method of any of the preceding claims, wherein the psilocybin analog is administered at a sub-hallucinogenic concentration.

4. The method of any of the preceding claims, wherein the psilocybin analog is a psilocybin analog of Table 1.

5. The method of any of the preceding claims, wherein the psilocybin analog is a psilocybin analog of Table 2.

6. The method of any of the preceding claims, wherein the psilocybin analog is baeocystin.

7. The method of claim 6, wherein the therapeutically effective amount of baeocystin ranges from about from about 9.5 mg/kg to about 10.5 mg/kg, from about 25 mg/kg to about 35 mg/kg, from about 45 mg/kg to about 55 mg/kg, or from about 165 mg/kg to about 175 mg/kg body weight of the subject.

8. The method of any of the preceding claims, wherein the psilocybin analog is norpsilocin.

9. The method of claim 8, wherein the therapeutically effective amount of norpsilocin ranges from about 0.25 mg/kg to about 0.35 mg/kg or from about 2.5 mg/kg to about 3.5 mg/kg body weight of the subject.

10. The method of any of the preceding claims, wherein the psilocybin analog is baeocystin, norpsilocin, or a combination of baeocystin and norpsilocin.

11. The method of claim 10, wherein the therapeutically effective amount of baeocystin, norpsilocin, or a combination of baeocystin and norpsilocin ranges from about 0.25 mg/kg to about 0.35 mg/kg or from about 2.5 mg/kg to about 3.5 mg/kg body weight of the subject.

12. A pharmaceutical composition for treating a psychological disorder, the composition comprising a psilocybin analog.

13. The pharmaceutical composition of claim 12, wherein the psilocybin analog is a psilocybin analog of Table 1.

14. The pharmaceutical composition of any one of the preceding claims, wherein the psilocybin analog is a psilocybin analog of Table 2.

15. The pharmaceutical composition of any one of the preceding claims, wherein the psilocybin analog is baeocystin.

16. The pharmaceutical composition of any one of the preceding claims, wherein the psilocybin analog is norpsilocin.

17. The pharmaceutical composition of any one of the preceding claims, wherein the psilocybin analog is baeocystin, norpsilocin, or a combination of baeocystin and norpsilocin.

18. A kit for treating a psychological disorder comprising one or more pharmaceutical compositions of claims 12 to 17 comprising a psilocybin analog.
